(19) United States
(12) Patent Application Publication
(10) Pub. No.: US 2025/0140346 A1
(43) Pub. Date: May 1, 2025

(54) SENSITIVITY OF TUMOR-INFORMED MINIMAL RESIDUAL DISEASE PANELS

(71) Applicant: Myriad Women's Health, Inc., South San Francisco, CA (US)

(72) Inventor: Ashley Acevedo, South San Francisco, CA (US)

(73) Assignee: Myriad Women's Health, Inc., South San Francisco, CA (US)

(21) Appl. No.: 18/930,340

(22) Filed: Oct. 29, 2024

Related U.S. Application Data
(60) Provisional application No. 63/546,466, filed on Oct. 30, 2023.

(51) Int. Cl. G16B 25/20; C12Q 1/6806; C12Q 1/6809; C12Q 1/6869; C12Q 1/6886; G16B 20/10; G16B 20/20; G16B 40/20

(52) U.S. Cl. CPC G16B 25/20; C12Q 1/6806; C12Q 1/6809; C12Q 1/6869; C12Q 1/6886; G16B 20/10; G16B 20/20; G16B 40/20; C12Q 2600/106

(57) ABSTRACT

Described herein are methods for improving sensitivity of tumor-informed MRD assays and panels by prioritizing sites to generate patient-specific panel of somatic variants based on expected variant allele frequency such that tumor somatic variants are overrepresented in cfDNA. Machine learning models trained with genomic data are also utilized to select the subset of somatic variants alone or in combination with an expected variant allele frequency.

### CROSS-REFERENCE TO RELATED APPLICATIONS

This application claims the benefit under 35 U.S.C. § 119 (e) of U.S. Provisional Patent Application No. 63/546,466, filed Oct. 30, 2023, the entire contents of which is incorporated herein by reference in its entirety.

### TECHNICAL FIELD

Described herein, are methods of improving the sensitivity and specificity of tumor-informed minimal residual disease (MRD) assays and panels.

### BACKGROUND

The following description of the background of the present technology is provided simply as an aid in understanding the present technology and is not admitted to describe or constitute prior art to the present technology.

The discovery of cell free deoxyribonucleic acid has promoted the non-invasive detection of alterations in genomic sequences that occur in various disease states. However, in some instances, e.g., cancer, the ability to determine the presence of disease by detecting disease-associated mutations has been hindered by the extremely low levels of cell free tumor DNA. Methods that allow for the accurate detection of disease-associated mutations remain desirable. In addition, there also remains a need for the determination of tumor fraction in pre- and post-treatment cancer patients.

### SUMMARY

The present disclosure provides methods for improving sensitivity of tumor-informed MRD assays and panels by prioritizing sites to generate patient-specific panel of somatic variants based on expected variant allele frequency such that tumor somatic variants are overrepresented in cell free DNA (cfDNA). Machine learning models trained with genomic data are also utilized to select the subset of somatic variants alone or in combination with an expected variant allele frequency.

In one aspect, the present disclosure provides methods of preparing a probe set for isolating circulating tumor DNA (ctDNA) from a sample, comprising: obtaining a tumor sample and a non-tumor sample from a cancer patient; sequencing DNA from the tumor sample and sequencing DNA from the non-tumor sample, thereby obtaining sequences of DNA from the tumor sample and sequences of DNA from the non-tumor sample; determining a set of somatic variants based on differences between sequences of DNA from the tumor sample and sequences of DNA from the non-tumor sample; calculating, using a computer processor, an expected variant allele frequency based on copy number and allele balance of each of the somatic variants; selecting a subset of somatic variants for which the expected variant allele frequency is above a reference threshold; and preparing a probe set comprising a plurality of oligonucleotides, wherein each oligonucleotide in the plurality of oligonucleotides comprises a nucleic acid sequence that is capable of hybridizing to a DNA fragment comprising one of the subset of somatic variants for which the expected variant allele frequency is above a reference threshold.

In an additional aspect, the present disclosure provides, methods of detecting circulating tumor DNA (ctDNA) in a sample, comprising: obtaining a tumor sample and a non-tumor sample from a cancer patient; sequencing DNA from the tumor sample and sequencing DNA from the non-tumor sample, thereby obtaining sequences of DNA from the tumor sample and sequences of DNA from the non-tumor sample; determining a set of somatic variants based on differences between sequences of DNA from the tumor sample and sequences of DNA from the non-tumor sample; calculating, using a computer processor, an expected variant allele frequency based on copy number and allele balance of each of the somatic variants; selecting a subset of somatic variants for which the expected variant allele frequency is above a reference threshold; and at a later time, obtaining at least one further sample of blood, plasma, or serum from the cancer patient; extracting cell-free DNA (cfDNA) from the at least one further sample; sequencing the cfDNA, thereby obtaining a sequencing library; and detecting the presence or absence of ctDNA in the sequencing library.

In some embodiment, the methods may further comprise enriching the extracted cfDNA by contacting the extracted cfDNA with a plurality of oligonucleotides, wherein each oligonucleotide in the plurality of oligonucleotides comprises a nucleic acid sequence that is capable of hybridizing to a DNA fragment comprising one of the somatic variants, thereby obtaining a ctDNA-enriched fraction.

In some embodiments, the methods further comprise repeating with a second, third, fourth, fifth, sixth, seventh, eight, ninth, or tenth further sample of blood, plasma, or serum at successive time points, at a later time, extracting cell-free DNA (cfDNA) from the at least one further sample; sequencing the cfDNA, thereby obtaining a sequencing library; and detecting the presence or absence of ctDNA in the sequencing library. In some embodiments, the method comprises enriching the cfDNA for ctDNA by contacting the extracted cfDNA with a plurality of oligonucleotides, wherein each oligonucleotide in the plurality of oligonucleotides comprises a nucleic acid sequence that is capable of hybridizing to a DNA fragment comprising one of the subset of somatic variants for which the expected variant allele frequency is above a reference threshold, thereby obtaining a ctDNA-enriched fraction.

In some embodiments, the repeating with a second, third, fourth, fifth, sixth, seventh, eight, ninth, or tenth further sample of blood, plasma, or serum at successive time points is while the patient is in remission.

In some embodiments, the repeating with a second, third, fourth, fifth, sixth, seventh, eight, ninth, or tenth further sample of blood, plasma, or serum at successive time points are while the patient is undergoing treatment for the cancer.

In some embodiments, the repeating with a second, third, fourth, fifth, sixth, seventh, eight, ninth, or tenth further sample of blood, plasma, or serum at successive time points coinciding with or prior to surgery; following, during, or prior to administration of chemotherapy; following, during, or prior to radiation therapy; following, during, or prior to administration of an immunotherapy; following, during, or prior to administration of a cell therapy; or following, during, or prior to administration of a biologic therapy.

In some embodiments, selecting a subset of somatic variants comprises subtracting an expected error rate form the expected variant allele frequency.

In a further aspect, the present disclosure provides, methods comprising: obtaining a tumor sample and a non-tumor sample from a cancer patient; sequencing DNA from the tumor sample and sequencing DNA from the non-tumor sample, thereby obtaining sequences of DNA from the tumor sample and sequences of DNA from the non-tumor sample; generating, by a computer device, a set of somatic variants based on differences between sequences of DNA from the tumor sample and sequences of DNA from the non-tumor sample; determining, based on a plurality of features of the set of somatic variants, a subset of somatic variants that are suitable for isolating circulating tumor DNA (ctDNA), wherein the plurality of features are selected by a machine learning model trained with genomic data; and providing an output indicating the subset of somatic variants that are suitable for isolating ctDNA.

In one aspect, the present disclosure provides, methods comprising: retrieving, by a processor, data associated with a tumor sample and a non-tumor sample from a cancer patient; generating, by the processor, a training data set comprising sequenced DNA from the tumor sample, sequenced DNA from the non-tumor sample, and a set of somatic variants based on differences between sequences of DNA from the tumor sample and sequences of DNA from the non-tumor sample; and training, by the processor, a machine learning model using the training dataset, such that the machine learning model is configured to ingest data associated with a second tumor sample and a second non-tumor sample from a patient and predict a subset of somatic variants that are suitable for isolating ctDNA for the patient.

In an additional aspect, the present disclosure provides, methods of preparing a probe set for isolating circulating tumor DNA (ctDNA) from a sample, comprising obtaining a tumor sample and a non-tumor sample from a cancer patient; sequencing DNA from the tumor sample and sequencing DNA from the non-tumor sample, thereby obtaining sequences of DNA from the tumor sample and sequences of DNA from the non-tumor sample; determining a set of somatic variants based on differences between sequences of DNA from the tumor sample and sequences of DNA from the non-tumor sample; calculating, using a computer processor, an expected variant allele frequency based on copy number and allele balance of each of the somatic variants; selecting a subset of somatic variants for which the expected variant allele frequency is above a reference threshold; and preparing a probe set comprising a plurality of oligonucleotides, wherein each oligonucleotide in the plurality of oligonucleotides comprises a nucleic acid sequence that is capable of hybridizing to a DNA fragment comprising one of the subset of somatic variants for which the expected variant allele frequency is above a reference threshold.

In some embodiments, selecting a subset of somatic variants comprises subtracting an expected error rate form the expected variant allele frequency.

In a further aspect, the present disclosure provides, methods of detecting circulating tumor DNA (ctDNA) in a sample, comprising: obtaining a tumor sample and a non-tumor sample from a cancer patient; sequencing DNA from the tumor sample and sequencing DNA from the non-tumor sample, thereby obtaining sequences of DNA from the tumor sample and sequences of DNA from the non-tumor sample; determining a set of somatic variants based on differences between sequences of DNA from the tumor sample and sequences of DNA from the non-tumor sample; selecting a subset of somatic variants that are suitable for isolating ctDNA by: (i) calculating, using a computer processor, an expected variant allele frequency based on copy number and allele balance of each of the somatic variants in the set of somatic variants, and selecting somatic variants for which the expected variant allele frequency is above a reference threshold, (ii) determining, based on a plurality of features of the set of somatic variants, a subset of somatic variants that are suitable for isolating ctDNA, wherein the plurality of features are selected by a machine learning model trained with genomic data, or a combination of (i) and (ii); and at a later time, obtaining at least one further sample of blood, plasma, or serum from the cancer patient; extracting cell-free DNA (cfDNA) from the at least one further sample; sequencing the cfDNA, thereby obtaining a sequencing library; and detecting the presence or absence of ctDNA in the sequencing library.

In some embodiments, selecting a subset of somatic variants comprises subtracting an expected error rate from the expected variant allele frequency.

In some embodiments, the genomic data comprises DNA sequencing data, whole genome sequencing data, whole exome sequencing data, targeted genomic sequencing data, cfDNA sequencing data, chromatin immunoprecipitation sequencing data, reference genome data, transcriptomics data, epigenomics data, or proteomics data.

In some embodiments, the methods further comprise repeating with a second, third, fourth, fifth, sixth, seventh, eight, ninth, or tenth further sample of blood, plasma, or serum at successive time points and at a later time, extracting cell-free DNA (cfDNA) from the at least one further sample; enriching the cfDNA for ctDNA (e.g., fragments that include target sequence corresponding to a tumor-specific somatic mutation or variant) by contacting the extracted cfDNA with a plurality of oligonucleotides, wherein each oligonucleotide in the plurality of oligonucleotides comprises a nucleic acid sequence that is capable of hybridizing to a DNA fragment comprising one of the subset of somatic variants that are suitable for isolating ctDNA, thereby obtaining a ctDNA-enriched fraction; sequencing the ctDNA-enriched fraction, thereby obtaining a sequencing library; and detecting the presence or absence of ctDNA in the sequencing library

In some embodiments, the repeating with a second, third, fourth, fifth, sixth, seventh, eight, ninth, or tenth further sample of blood, plasma, or serum at successive time points is while the patient is in remission.

In some embodiments, the repeating with a second, third, fourth, fifth, sixth, seventh, eight, ninth, or tenth further sample of blood, plasma, or serum at successive time points is while the patient is undergoing treatment for the cancer.

In some embodiments, the repeating with a second, third, fourth, fifth, sixth, seventh, eight, ninth, or tenth further sample of blood, plasma, or serum at successive time points coinciding with or prior to surgery; following, during, or prior to administration of chemotherapy; following, during, or prior to radiation therapy; following, during, or prior to administration of an immunotherapy; following, during, or prior to administration of a cell therapy; or following, during, or prior to administration of a biologic therapy.

In one aspect, the present disclosure provides, methods comprising: retrieving, by a processor, data associated with a tumor sample and a non-tumor sample from a cancer patient, the data comprising DNA sequences from the tumor sample and DNA sequences from the non-tumor sample; calculating, by the processor, a set of somatic variants based on differences between sequences of DNA from the tumor sample and sequences of DNA from the non-tumor sample; calculating, by the processor using a first computer model, an expected allele frequency of each of the somatic variants; calculating, by the processor using a second computer model, at least one valid target site associated with at least one somatic variant; and selecting, by the processor, a subset of somatic variants in accordance with an output of the first computer model and the second computer model.

In some embodiments, the subset of somatic variants are selected in accordance with at least a portion of the set of somatic variants having an expected variant allele frequencies satisfying a threshold and are suitable for isolating ctDNA for the patient.

In some embodiments, the methods may further comprise enriching the extracted cfDNA by contacting the extracted cfDNA with a plurality of oligonucleotides, wherein each oligonucleotide in the plurality of oligonucleotides comprises a nucleic acid sequence that is capable of hybridizing to a DNA fragment comprising one of the somatic variants, thereby obtaining a ctDNA-enriched fraction. In some embodiments, enriching the extracted cfDNA comprises contacting the extracted cfDNA with s plurality of oligonucleotides, wherein each oligonucleotide in the first plurality of oligonucleotides comprises a nucleic acid sequence that is capable of hybridizing to a DNA fragment comprising one of the somatic variants and a corresponding control site, wherein the corresponding control site for each somatic variant is located within 20 bases of the somatic variant on the DNA fragment, and wherein the corresponding control site optionally comprises a reference base that is that same as the base of the somatic variant. For the purposes of any embodiment involving enriching cfDNA prior to sequencing, the enriching comprises (i) hybrid capture-based enrichment, (ii) PCR-target enrichment, or (iii) on-sequencer enrichment.

In some embodiments, the sequencing comprises whole genome sequencing or targeted sequencing. In some embodiments, the targeted sequencing comprises sequencing of introns, exons, intergenic regions, or a combination thereof.

In some embodiments, the subset of somatic variants comprises at least 10, at least 50, at least 100, at least 150, at least 200, at least 250, at least 500, at least 750, at least 1000, at least 1100, at least 1200, at least 1300, at least 1400, at least 1500, at least 1600, at least 1700, at least 1800, at least 1900, or at least 2000 tumor-specific somatic mutations.

In some embodiments, the subset of somatic variants comprises one or more somatic mutations selected from SNVs, insertions, deletions, and translocations.

In some embodiments, the methods further comprise determining a tumor fraction. In some embodiments, a tumor fraction of zero indicates the absence of the tumor in the patient.

In some embodiments, the tumor sample comprises a solid tumor biopsy or a fluid sample.

In some embodiments, the fluid sample is selected from blood, blood plasma, blood serum, urine, saliva, and cerebral spinal fluid (CSF).

In some embodiments, the non-tumor sample comprises a tissue sample matched to a tissue of origin of the tumor sample.

In some embodiments, the non-tumor sample comprises a fluid sample selected from a buffy coat sample, blood, blood plasma, blood serum, urine, saliva, and cerebral spinal fluid (CSF).

In some embodiments, the patient has completed at least one cancer treatment prior to obtaining the tumor sample and the non-tumor sample.

In some embodiments, the cancer treatment is selected from chemotherapy, radiotherapy, surgery, immunotherapy, cell therapy, or biologic therapy.

In some embodiments, the tumor is selected from adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor.

### BRIEF DESCRIPTION OF THE FIGURES

FIG. 1: shows a block diagram illustrating an example computer environment for implementing methods and processes described herein, according to an embodiment.

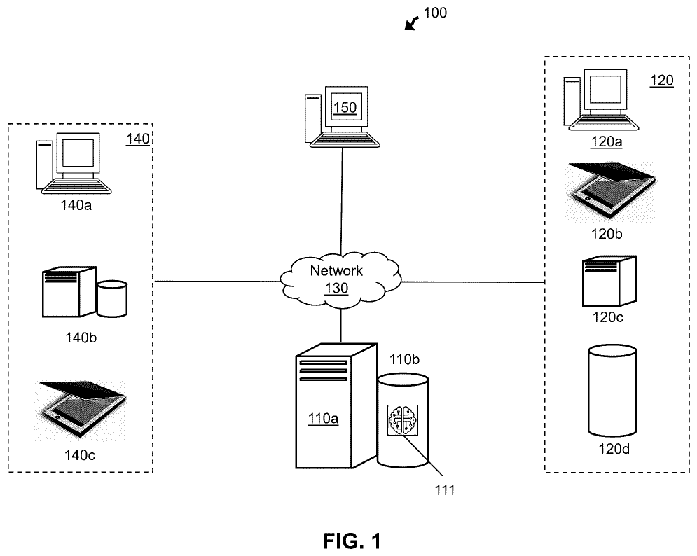

FIG. 2: Shows the number of somatic variant calls for 13 samples of approximately 15,000 targets classified as true positive (TP) or false positive (FP) variant calls.

FIG. 3: Shows the difference in positive predictive value (PPV) before and after inclusion of the random forest (RF) classifier based on the level of confidence of each somatic call (measured by number of callers in the ensemble that identified the target variant). The left-most points are the PPV without the RF classification. The right-most points are PPV with RF classification. Size of the right-most point corresponds to the fraction of the original target variants that were classified as true positive (TP) by the RF classifier.

FIG. 4: Shows the difference in positive predictive value (PPV) before and after inclusion of the random forest (RF) classifier based on the amount of tumor sequence depth obtained for each somatic variant call. The left-most points are the PPV without the RF classification. The right-most points are PPV with RF classification. Size of the right-most point corresponds to the fraction of the original target variants that were classified as true positive (TP) by the RF classifier.

FIG. 5: Shows the relationship of GC content of a target region spanning 41 bp with respect to target depth and positive predictive value (PPV). Relative target depth, the depth of a target relative to other targets in the panel, are the top-most points with error bars. PPV are the light grey points connected by lines. Effective depth, relative depth * PPV, are the bottom points with error bars.

FIG. 6: Shows the simulation of effective panel depth for “random” (all targets) on the left and panels “optimized” for GC content and positive predictive value (PPV) on the right.

FIG. 7: Shows the relationship of mappability, the number or blast hits, of a target region spanning 101 bp with respect to target depth and positive predictive value (PPV). Relative target depth, the depth of a target relative to other targets in the panel, are the top-most points with error bars. PPV is shown in light grey connected by lines. Effective depth, relative depth * PPV, are the bottom points with error bars.

FIG. 8: Shows the relationship of sequence entropy of a target region spanning 21 bp with respect to target depth and positive predictive value (PPV). Relative target depth, the depth of a target relative to other targets in the panel, are the top-most points with error bars. PPV is shown in light grey connected by lines. Effective depth, relative depth * PPV, are the bottom points with error bars.

FIG. 9: Shows the simulation of effective panel depth for “random” (all targets) on the left and panels “optimized” for mappability and positive predictive value (PPV) on the right.

FIG. 10: Shows the simulation of effective panel depth for “random” (all targets) on the left and panels “optimized” for sequence entropy and positive predictive value (PPV) on the right.

FIG. 11: Shows effect of mappability, entropy, and GC content, when considered with positive predictive value (PPV) either alone or in combination on effective panel depth.

### DETAILED DESCRIPTION

The invention will now be described in detail by way of reference only using the following definitions and examples. All patents and publications, including all sequences disclosed within such patents and publications, referred to herein are expressly incorporated by reference.

### I. Definitions

As used herein, the singular forms “a,” “an,” and “the” include the plural reference unless the context clearly dictates otherwise.

Reference to “about” a value or parameter herein includes (and describes) variations that are directed to that value or parameter per se. The term “about” is used herein to mean plus or minus ten percent (10%) of a value. For example, “about 100” refers to any number between 90 and 110.

It is understood that aspects and variations of the invention described herein include “consisting” and/or “consisting essentially of” aspects and variations.

Numeric ranges are inclusive of the numbers defining the range.

Unless otherwise indicated, nucleic acids are written left to right in 5′ to 3′ orientation; amino acid sequences are written left to right in amino to carboxy orientation, respectively.

The term “mutation” herein refers to a change introduced into a reference sequence, including, but not limited to, substitutions, insertions, deletions (including truncations) relative to the reference sequence. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms (SNPs), single nucleotide variant (SNV), multiple nucleotide polymorphisms, insertions (e.g., insertion of one or more nucleotides at a locus but less than the entire locus), multiple nucleotide changes, deletions (e.g., deletion of one or more nucleotides at a locus), inversions (e.g., reversal of a sequence of one or more nucleotides), an genomic rearrangements (e.g., deletions, duplications, inversions, and translocations). In some embodiments, the reference sequence is a parental sequence. In some embodiments, the reference sequence is a reference human genome, e.g., h19. In some embodiments, the reference sequence is derived from a non-cancer (or non-tumor) sequence. In some embodiments, the mutation is inherited. In some embodiments, the mutation is spontaneous or de nova. In some embodiments, the mutation is a “somatic” mutation or variant.

The term “somatic variant” or “somatic mutation” herein refers to a variant arising after conception, in non-germline DNA of an individual. Somatic variants may include single-nucleotide variants (SNVs), multi-nucleotide variants, insertions and deletions (e.g., indel variants), and genomic rearrangements for example. The terms “somatic variant” and “somatic mutation” are used interchangeably herein. In some embodiments, the terms “somatic variant” or “somatic mutation” refers to a collection of somatic variants that are specific to a patient.

The term “patient-specific panel” or “a set of somatic variants” herein refers to a collection of sequences comprising somatic mutations that are specific to a patient, or markers that distinguish between two or more individuals. A signature panel may distinguish one sample from another.

The term “a subset of somatic variants” or “subset panel” herein refers to a subset of somatic variants of the patient-specific panel or set of somatic variants. In some embodiments, the subset is based on a plurality of features selected from a machine learning model and/or an expected variant allele frequency reference threshold.

The term “machine learning model” herein refers to any computer model that uses artificial intelligence techniques to train itself using a training dataset, such that it can ingest new data (at the inference phase) and predict an outcome using patterns and commonalities learned during the training phase.

The term “tumor fraction” herein refers to the proportion of circulating cell-free tumor DNA (ctDNA) relative to the total amount of cell-free DNA (cfDNA). Tumor fraction may be indicative of the size of the tumor.

The term “genomic DNA” refers to DNA of a cellular genome. The genomic DNA can be cellular, i.e., contained within a cell, or it can be cell free.

The term “sample” herein refers to any substance containing or presumed to contain nucleic acid. The sample can be a biological sample obtained from a subject or patient. The nucleic acids can be RNA, DNA, e.g., genomic DNA. In some embodiments, the biological sample is a biological fluid sample. The fluid sample can be whole blood, plasma, serum, ascites, cerebrospinal fluid, sweat, urine, tears, saliva, buccal sample, cavity rinse, or organ rinse. The fluid sample can be an essentially cell-free liquid sample (e.g., plasma, serum, sweat, urine, tears, etc.). In other embodiments, the biological sample is a solid biological sample, e.g., feces or tissue biopsy, such as a tumor biopsy. In some embodiment, the sample is a tumor sample. In some embodiments, the sample is a non-tumor sample. A “sample” may include, but is not limited to, tissue, blood, plasma, saliva, urine, semen, amniotic fluid, oocytes, skin, hair, feces, cheek swabs, or pap smear lysate from an individual. In some embodiments, the sample is blood, plasma, or serum.

The term “target sequence” herein refers to a selected target polynucleotide, e.g., a sequence present in a cfDNA molecule, whose presence, amount, and/or nucleotide sequence, or changes in these, are desired to be determined. Target sequences are interrogated for the presence or absence of a somatic variant. The target polynucleotide can be a region of gene associated with a disease. In some embodiments, the region is an exon. The disease can be cancer.

The terms “anneal,” “hybridize,” or “bind,” can refer to two polynucleotide sequences, segments or strands, and can be used interchangeably and have the usual meaning in the art. Two complementary sequences (e.g., DNA and/or RNA) can anneal or hybridize by forming hydrogen bonds with complementary bases to produce a double-stranded polynucleotide or a double-stranded region of a polynucleotide.

The term “marker” or “segregating marker” refers to a moiety that is used to discriminate between two or more samples, e.g., two or more individuals or tissues. A marker may be a nucleic acid (e.g., a gene), small molecule, peptide, fatty acid, metabolite, protein, lipid, etc. A marker may be a mutation. A marker may be a synthetic nucleic acid. A marker or set of markers may define a genetic signature of an entity, e.g., an individual, relative to a second nucleic acid, e.g., a reference nucleic acid sequence.

The terms “treat,” “treatment,” and “treating” refer to the reduction or amelioration of the progression, severity, and/or duration of a proliferative disorder e.g., cancer, or the amelioration of a proliferative disorder resulting from the administration of one or more therapies.

As used herein, the term “barcode” (also termed single molecule identifier or SMI) refers to a known nucleic acid sequence that allows some feature of a polynucleotide with which the barcode is associated to be identified. In some embodiments, the feature of the polynucleotide to be identified is the sample from which the polynucleotide is derived. In some embodiments, barcodes are about or at least about 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some polynucleotides are of different lengths than barcodes associated with other polynucleotides. In general, barcodes are of sufficient length and include sequences that are sufficiently different to allow the identification of samples based on barcodes with which they are associated. In some embodiments, a barcode, and the sample source with which it is associated, can be identified accurately after the mutation, insertion, or deletion of one or more nucleotides in the barcode sequence, such as the mutation, insertion, or deletion of 1, 2, 3, 4, 5, 6, 7, 8, 9, 10, or more nucleotides. In some embodiments, each barcode in a plurality of barcodes differ from every other barcode in the plurality at least three nucleotide positions, such as at least 3, 4, 5, 6, 7, 8, 9, 10, or more nucleotide positions. A plurality of barcodes may be represented in a pool of samples, each sample including polynucleotides comprising one or more barcodes that differ from the barcodes contained in the polynucleotides derived from the other samples in the pool. Samples of polynucleotides including one or more barcodes can be pooled based on the barcode sequences to which they are joined, such that all four of the nucleotide bases A, G, C, and T are approximately evenly represented at one or more positions along each barcode in the pool (such as at 1, 2, 3, 4, 5, 6, 7, 8, or more positions, or all positions of the barcode).

The term “small nucleotide polymorphism” or “SNP” refers to a single-nucleotide variant (SNV), a multi-nucleotide variant (MNV), or an indel variant about 100 base pairs or less.

The term “multi-nucleotide variant” or “MNV” herein refers to a variant having 2 or more adjacent nucleotide changes.

The term “copy number variant,” “CNV,” or “copy number” refers to any duplication or deletion of a genomic segment. In some embodiments, the copy number is the copy number of each somatic variant in the set of somatic variants.

The term “allele balance” refers to a ratio of a variant allele to a reference allele. In some embodiments, the variant allele is a variant allele from each somatic variant in the set of somatic variants.

The term “expected error rate” refers to a prior estimate for errors based on the identity of a mutation (e.g., C>T. T>A).

The term “derived from” encompasses the terms “originated from,” “obtained from,” “obtainable from,” “isolated from,” and “created from,” and generally indicates that one specified material (e.g., a biological sample) finds its origin in another specified material or individual or has features that can be described with reference to another specified material.

The term “library” or “sequencing library” herein refers to a collection or plurality of template molecules, i.e., target DNA duplexes, which share common sequences at their 5′ ends and common sequences at their 3′ ends. Use of the term “library” to refer to a collection or plurality of template molecules should not be taken to imply that the templates making up the library are derived from a particular source, or that the “library” has a particular composition. By way of example, use of the term “library” should not be taken to imply that the individual templates within the library must be of different nucleotide sequence or that the templates must be related in terms of sequence and/or source. In general, the term “sequencing library” herein refers to DNA that is processed for sequencing, e.g., using massively parallel methods, e.g., NGS. The DNA may optionally be amplified to obtain a population of multiple copies of processed DNA, which can be sequenced by NGS.

The term “Next Generation Sequencing” or “NGS” refers to sequencing methods that allow for massively parallel sequencing of clonally amplified and of single nucleic acid molecules during which a plurality, e.g., millions, of nucleic acid fragments from a single sample or from multiple different samples are sequenced in unison. Non-limiting examples of NGS include sequencing-by-synthesis, sequencing-by-ligation, real-time sequencing, and nanopore sequencing.

The term “sequence read” or simply “read” herein refers to sequence information of a nucleic acid fragment obtained through a sequencing assay, such as a next generation sequencing (NGS) assay. In some embodiments, a sequence read refers to data representing a sequence of nucleotide bases that were measured using a clonal sequencing method. Clonal sequencing may produce sequence data representing single, or clones, or clusters of one original DNA molecule. A sequence read may also have associated quality score at each base position of the sequence indicating the probability that nucleotide has been called correctly.

The term “mapping a sequence read” herein refers to the process of determining a sequence read's location of origin in the genome sequence of a particular organism. The location of origin of sequence reads is based on similarity of nucleotide sequence of the read and the genome sequence.

The term “preferential enrichment” of DNA that corresponds to a locus, or preferential enrichment of DNA at a locus, refers to any method that results in the percentage of molecules of DNA in a post-enrichment DNA mixture that correspond to the locus being higher than the percentage of molecules of DNA in the pre-enrichment DNA mixture that correspond to the locus. The method may involve selective amplification of DNA molecules that correspond to a locus. The method may involve removing DNA molecules that do not correspond to the locus. The method may involve a combination of methods. The degree of enrichment is defined as the percentage of molecules of DNA in the post-enrichment mixture that correspond to the locus divided by the percentage of molecules of DNA in the pre-enrichment mixture that correspond to the locus. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

The term “amplification,” with respect to nucleic acid sequences, herein refers to methods that increase the representation of a population of nucleic acid sequences in a sample. Copies of a particular target nucleic acid sequence generated in vitro in an amplification reaction are called “amplicons” or “amplification products”. Amplification may be exponential or linear. A target nucleic acid may be DNA (such as, for example, genomic DNA, ctDNA, cfDNA, and cDNA) or RNA. While the exemplary methods described hereinafter relate to amplification using polymerase chain reaction (PCR), numerous other methods such as isothermal methods, rolling circle methods, etc., are available to the skilled artisan. The skilled artisan will understand that these other methods may be used either in place of, or together with, PCR methods. See, e.g., Saiki, “Amplification of Genomic DNA” in PCR PROTOCOLS, Innis et al., Eds., Academic Press, San Diego, CA 1990, pp 13-20; Wharam, et al., Nucleic Acids Res. 29 (11): E54-E54 (2001).

The term “selective amplification” herein refers to a method that increases the number of copies of a particular molecule of DNA, or molecules of DNA that correspond to a particular region of DNA. It may also refer to a method that increases the number of copies of a particular targeted molecule of DNA, or targeted region of DNA more than it increases non-targeted molecules or regions of DNA. Selective amplification may be a method of preferential enrichment.

The term “direct amplification” herein refers to a nucleic acid amplification reaction in which the target nucleic acid is amplified from the sample without prior purification, extraction, or concentration.

The term “amplification mixture” herein refers to a mixture of reagents that are used in a nucleic acid amplification reaction, but does not contain primers or sample. An amplification mixture comprises a buffer, dNTPs, and a DNA polymerase. An amplification mixture may further comprise at least one of MgCl₂, KCl, nonionic and ionic detergents (including cationic detergents). In general, amplification methods disclosed herein with include an amplification mixture. The term “amplification master mix” refers to an amplification mixture, primers, and/or probes for amplifying one or more target nucleic acids, but does not contain the sample to be amplified. The term “reaction-sample mixture” herein refers to a mixture containing amplification master mix and a sample.

The term “multiplex PCR” herein refers to the simultaneous generation of two or more PCR products or amplicons within the same reaction vessel. Similarly, a “2-plex PCR” refers to the simultaneous generation of two PCR products or amplicons within the same reaction vessel. Each PCR product is primed using a distinct primer pair. A multiplex reaction may further include specific probes for each product that are labeled with different detectable moieties.

The term “universal priming sequence” refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences are typically not related to the target sequences.

The term “universal adapters” or “ligation adaptors” or “library tags” are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.

The term “targeting” herein refers to a method used to selectively amplify or otherwise preferentially enrich those molecules of DNA that correspond to a set of loci, in a mixture of DNA.

The term “primer” herein refers to an oligonucleotide, whether occurring naturally or produced synthetically, which is capable of acting as a point of initiation of nucleic acid synthesis when placed under conditions in which synthesis of a primer extension product which is complementary to a nucleic acid strand is induced, e.g., in the presence of four different nucleotide triphosphates and a polymerase enzyme, e.g., a thermostable enzyme, in an appropriate buffer (“buffer” includes pH, ionic strength, cofactors, etc.) and at a suitable temperature. The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the polymerase, e.g., thermostable polymerase enzyme. The exact lengths of a primer will depend on many factors, including temperature, source of primer and use of the method. For example, depending on the complexity of the target sequence, the oligonucleotide primer typically contains 15-25 nucleotides, although it may contain more or few nucleotides. Short primer molecules generally require colder temperatures to form sufficiently stable hybrid complexes with template.

A “hybrid capture probe” herein refers to any nucleic acid sequence, possibly modified, that is generated by various methods such as PCR or direct synthesis and intended to be complementary to one strand of a specific target DNA sequence in a sample. The exogenous hybrid capture probes may be added to a prepared sample and hybridized through a denature-reannealing process to form duplexes of exogenous-endogenous fragments. These duplexes may then be physically separated from the sample by various means.

A “spacer” may consist of a repeated single nucleotide (e.g., 1, 2, 3, 4, 5, 6, 7, 8, 9, 10, or more of the same nucleotide in a row), or a sequence of 2, 3, 4, 5, 6, 7, 8, 9, 10, or more nucleotides repeated 1, 2, 3, 4, 5, 6, 7, 8, 9, 10, or more times. A spacer may comprise or consist of a specific sequence, such as a sequence that does not hybridize to any target sequence in a sample. A spacer may comprise or consist of a sequence of randomly selected nucleotides.

The phrases “substantially similar” and “substantially identical” in the context of at least two nucleic acids typically means that a polynucleotide includes a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) polynucleotide or polypeptide. Sequence identity may be determined using known programs such as BLAST, ALIGN, and CLUSTAL using standard parameters. (See, e.g., Altshul et al. (1990) J. Mol. Biol. 215:403-410; Henikoff et al. (1989) Proc. Natl. Acad. Sci. 89:10915; Karin et al. (1993) Proc. Natl. Acad. Sci. 90:5873; and Higgins et al. (1988) Gene 73:237). Software for performing BLAST analyses is publicly available through the National Center for Biotechnology Information. Also, databases may be searched using FASTA (Person et al. (1988) Proc. Natl. Acad. Sci. 85:2444-2448.) In some embodiments, substantially identical nucleic acid molecules hybridize to each other under stringent conditions (e.g., within a range of medium to high stringency).

The term “tag” refers to a detectable moiety that may be one or more atom(s) or molecule(s), or a collection of atoms and molecules. A tag may provide an optical, fluorescent, electrochemical, magnetic, or electrostatic (e.g., inductive, capacitive) signature.

The term “tagged nucleotide” herein refers to a nucleotide that includes a tag (or tag species) that is coupled to any location of the nucleotide including, but not limited to a phosphate (e.g., terminal phosphate), sugar or nitrogenous base moiety of the nucleotide. Tags may be one or more atom(s) or molecule(s), or a collection of atoms and molecules. A tag may provide an optical, electrochemical, magnetic, or electrostatic (e.g., inductive, capacitive) signature.

As used herein, the term “target polynucleotide” refers to a nucleic acid molecule or polynucleotide in a population of nucleic acid molecules having a target sequence to which one or more oligonucleotides are designed to hybridize. “Target polynucleotide” may be used to refer to a double-stranded nucleic acid molecule that includes a target sequence on one or both strands, or a single-stranded nucleic acid molecule including a target sequence, and may be derived from any source of or process for isolating or generating nucleic acid molecules. A target polynucleotide may include one or more (e.g., 1, 2, 3, 4, 5, 6, 7, 8, 9, 10, or more) target sequences, which may be the same or different. In general, different target polynucleotides include different sequences, such as one or more different nucleotides or one or more different target sequences.

The term “template DNA molecule” herein refers to a strand of a nucleic acid from which a complementary nucleic acid strand is synthesized by a DNA polymerase, for example, in a primer extension reaction.

A “portion adjacent to a region of interest” refers to a sequence that is immediately proximal to a region of interest. Reference to a “portion of or adjacent to a region of interest” refers to a sequence that 1) is entirely within the region of interest, 2) is entirely outside but immediately proximal to the region of interest, or 3) includes a contiguous sequence from within and immediately proximal to the region of interest. Reference to a “sequence that is substantially complementary to a portion of or adjacent to a region of interest” refers to 1) a sequence that is substantially complementary to a sequence entirely within the region of interest, 2) a sequence substantially complementary to a sequence entirely outside but immediately proximal to the region of interest, or 3) a sequence that is substantially complementary to a contiguous sequence from with and immediately proximal to the region of interest.

“Noisy Genetic Data” herein refers to genetic data with any of the following: allele dropouts, uncertain base pair measurements, incorrect base pair measurements, missing base pair measurements, uncertain measurements of insertions or deletions, uncertain measurements of chromosome segment copy numbers, spurious signals, missing measurements, other errors, or combinations thereof.

“Confidence” herein refers to the statistical likelihood that the called SNP, SNV, variant, copy number, etc. correctly represents the real genetic state of the individual.

### II. Minimal Residual Disease Detection

The goal of a minimum residual disease (MRD) assay is to detect and/or quantify circulating tumor DNA (ctDNA) so researchers and clinicians can detect recurrence early and monitor the progress of the disease through treatment. In general, an MRD assay will rely on a patient-specific and tumor-specific panel (i.e., a “signature panel” or “a set of somatic variants”) for assessing the presence of ctDNA in a patient sample. The signature panel can be prepared with the general steps of (1) profiling a tumor or cancer sample from a patient, and (2) identifying a subset of somatic mutations to target, and, at one or more later time points, (3) taking a subsequent sample from the patient, (4) enriching cell-free DNA (cfDNA) for the target somatic mutation sites, and (5) determining or estimating the ctDNA content of the cfDNA given the tumor profile and sequencing data.

More specifically, preparing the patient-specific and tumor-specific panel (i.e., a “signature panel” or “a set of somatic variants”) may comprise, for example, (a) obtaining a tumor sample and a non-tumor sample from a cancer patient; (b) sequencing DNA from the tumor sample (e.g., genomic DNA) and sequencing DNA from the non-tumor sample (e.g., cfDNA), thereby obtaining sequences DNA or sequence reads from the tumor sample and the non-tumor sample; and (c) comparing the sequences of the tumor sample and the non-tumor sample to determine any tumor-specific somatic mutations that are present in the sequences of DNA from the tumor sample but not present in the sequences of DNA from the non-tumor sample. Sequencing of the DNA from the tumor sample and non-tumor sample may comprise whole genome sequencing or various types of targeted sequencing, such as whole exome sequencing.

This comparison of the tumor and non-tumor sequences can be performed by, for example, aligning the sequences of DNA from the tumor sample (e.g., genomic DNA) to a reference human genome that is not from the patient and aligning the sequences of DNA (e.g., cfDNA) from the non-tumor sample to the reference genome that is not from the patient. The reference genome can be, for example, a publicly available human genome assembly, such as hg18, hg19, GRCh38.p14, GRCh37.p13, or other assemblies from the Genome Reference Consortium. Alternatively, the comparison of the tumor and non-tumor sequences can be performed by, for example, aligning the sequences of DNA (e.g., genomic DNA) from the tumor sample to sequences of DNA (e.g., cfDNA) from the non-tumor sample. With either approach, the skilled artisan is able to detect and identify tumor-specific somatic mutations that are present in the tumor sample but not in the non-tumor sample.

The tumor sample may be a solid tumor sample, such as a biopsy or other tissue sample, or a liquid sample or a fluid sample, such as blood (in the case of a hematological cancer) or specific fractions of blood. The non-tumor sample may be tissue-matched with the tumor sample or it may be from a different tissue. For example, the non-tumor sample may be selected from a healthy (i.e., non-cancerous or non-tumor) tissue sample, blood or specific fractions of blood such as buffy coat, leukocytes, fibroblast, or any other biological sample comprising cfDNA or genomic DNA. In some embodiment, the tumor sample comprises a tumor biopsy or fluid sample. In some embodiments, the fluid sample is selected from blood, blood plasma, blood serum, urine, saliva, and cerebral spinal fluid (CSF). In some embodiment, the non-tumor sample comprises a tissue sample matched to a tissue of origin of the tumor sample. In some embodiments, the non-tumor sample comprises a fluid sample selected from a buffy coat sample, blood, blood plasma, blood serum, urine, saliva, and cerebral spinal fluid (CSF).

Once a patient-specific and tumor-specific panel (i.e., a “signature panel” or “a set of somatic variants”) has been established, such a signature panel can be used to enrich ctDNA (i.e., fragments that include a tumor-specific somatic mutation or variant) in subsequent samples taken from the cancer patient. The subsequent samples may be taken from a patient at various time points during the course of treatment or during a period of remission. For example, after a surgical removal of a tumor, the tumor may be profiled as described herein to determine tumor-specific somatic mutations, and at one or more subsequent time points a subsequent sample may be taken from the subject to search for the presence of any ctDNA comprising any one of the identified tumor-specific somatic mutations. The detection or presence of ctDNA comprising a tumor-specific somatic mutation may be indicative of cancer recurrence. Additionally or alternatively, similar assessment can be performed throughout the course of a patient's treatment (e.g., with chemotherapy, radiation, immunotherapy, cell therapy, etc.) to detect or quantify ctDNA and determine whether the amount of ctDNA is increasing or decreasing, as this may be indicative of responsiveness to the therapy. Accordingly, assessment of a subsequent sample may be repeated 1, 2, 3, 4, 5, 6, 7, 8, 9, or 10 or more times throughout the course of a patient's remission or treatment. The assessment of a subsequent sample may be repeated monthly, every other month, once every three months, once every four months, once every five months, once every six months, once every seven months, once every eight months, once every nine months, once every ten months, once every eleven months, or annually.

The type of sample used for the one or more subsequent samples is generally a blood sample, a plasma sample, or a serum sample, but any biological sample that contains cfDNA and potential contains ctDNA would be acceptable. In some embodiments, the one or more subsequent samples are cell-free samples.

Enrichment of ctDNA (e.g., fragments that include a target sequence corresponding to a tumor-specific somatic mutation or variant) in the one or more subsequent samples can be performed by methods including, but not limited to, hybrid capture-based enrichment, PCR-target enrichment, or on-sequencer enrichment. Briefly, enrichment may comprise extracting cfDNA from a subsequent sample taken from the cancer patient and contacting the extracted cfDNA with a plurality of oligonucleotides (i.e., oligonucleotide probes), wherein each oligonucleotide in the plurality of oligonucleotides comprises a nucleic acid sequence that is capable of hybridizing to a cfDNA fragment comprising one of the tumor-specific somatic mutation sequences identified by comparing the sequences of the patients tumor DNA and non-tumor DNA. In some embodiments, the nucleic acid sequence is capable of hybridizing 1 or more nucleotide bases upstream or downstream of the tumor-specific somatic mutation sequences. Thus, enrichment may utilize a set of oligonucleotide probes to selectively enrich ctDNA that may be in the subsequent sample by binding to previously identified tumor-specific somatic mutation sequences.

A signature panel, a set of somatic variants, or a subset of somatic variants may comprise 10-5000 tumor-specific somatic mutations. For example, a signature panel may comprise 10-4000, 10-3000, 10-2500, 10-2000, 10-1500, 10-1000, 10-950, 10-900, 10-850, 10-800, 10-750, 10-700, 10-650, 10-600, 10-550, 10-500, 50-5000, 50-4000, 50-3000, 50-2500, 50-2000, 50-1500, 50-1000, 50-950, 50-900, 50-850, 50-800, 50-750, 50-700, 50-650, 50-600, 50-550, 50-500, 100-5000, 100-4000, 100-3000, 100-2500, 100-2000, 100-1500, 100-1000, 100-950, 100-900, 100-850, 100-800, 100-750, 100-700, 100-650, 100-600, 100-550, 100-500, 200-5000, 200-4000, 200-3000, 200-2500, 200-2000, 200-1500, 200-1000, 200-950, 200-900, 200-850, 200-800, 200-750, 200-700, 200-650, 200-600, 200-550, 200-500, 300-5000, 300-4000, 300-3000, 300-2500, 300-2000, 300-1500, 300-1000, 300-950, 300-900, 300-850, 300-800, 300-750, 300-700, 300-650, 300-600, 300-550, 300-500, 400-5000, 400-4000, 400-3000, 400-2500, 400-2000, 400-1500, 400-1000, 400-950, 400-900, 400-850, 400-800, 400-750, 400-700, 400-650, 400-600, 400-550, 400-500, 500-5000, 500-4000, 500-3000, 500-2500, 500-2000, 500-1500, 500-1000, 500-950, 500-900, 500-850, 500-800, 500-750, 500-700, 500-650, 500-600, or 500-550 tumor-specific somatic mutations. In some embodiments, a signature panel, a set of somatic variants or a subset of somatic variants may comprise or consist of about 10, about 20, about 30, about 40, about 50, about 75, about 100, about 150, about 200, about 250, about 300, about 350, about 400, about 450, about 500, about 550, about 600, about 650, about 700, about 750, about 800, about 850, about 900, about 950, about 1000, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, or about 2000 or more tumor-specific somatic mutations. In some embodiments, a signature panel, a set of somatic variants, or a subset of somatic variants may comprise at least 10, at least 20, at least 30, at least 40, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1150, at least 1200, at least 1250, at least 1300, at least 1350, at least 1400, at least 1450, at least 1500, at least 1550, at least 1600, at least 1650, at least 1700, at least 1750, at least 1800, at least 1850, at least 1900, at least 1950, or at least 2000 tumor-specific somatic mutations. The tumor-specific somatic mutations may be in introns, exons, or a combination thereof. In some embodiments, the tumor-specific mutations may be one or more somatic mutations selected from SNVs, insertions, deletions and translocations.

After enrichment or concurrently with enrichment of ctDNA (e.g., fragments that a include target sequence corresponding to a tumor-specific somatic mutation or variant), the enriched DNA is sequenced. This sequencing may be performed by, for example Next Generation Sequencing (NGS). Deep sequencing may allow for more sensitive detection, and so the depth of the sequencing may be at least 50×, at least 100×, at least 150×, at least 200×, at least 250×, at least 300×, at least 350×, at least 400×, at least 450×, at least 500×, at least 550×, at least 600×, at least 650×, at least 700×, at least 750×, at least 800×, at least 850×, at least 900×, at least 950×, or at least 1000×. In other words, the depth of the sequencing may be about 50×, about 100×, about 150×, about 200×, about 250×, about 300×, about 350×, about 400×, about 450×, about 500×, about 550×, about 600×, about 650×, about 700×, about 750×, about 800×, about 850×, about 900×, about 950×, or about 1000×. The detection sensitivity of the disclosed methods may be about 20 to about 50 ctDNA fragments comprising one or more of the set of somatic mutations in the fluid sample per a total background of about 500,000 cfDNA fragments.

The disclosed methods may be used for tracking and assessing recurrence in any cancer patient. For example, the cancer patient may have a cancer selected from, but not limited to, adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor. In some embodiments, the cancer may be a blood borne or hematological cancer such as leukemia or lymphoma.

The disclosed MRD assay, specifically the obtaining and testing of subsequent samples from a cancer patient, may be repeated one or more times following completion of a cancer treatment; one or more times while the cancer patient is in remission; one or more times coinciding with or prior to surgery; following, during, or prior to administration of chemotherapy; following, during, or prior to radiation therapy; following, during, or prior to immunotherapy; or following, during, or prior to cell therapy; or following, during, or prior to administration of a biologic therapy. The disclosed MRD assay may also be repeated at times prior to, coinciding with, and/or following an imaging test, such as a PET scan, a PET/CT scan, an MRI, or an X-ray.

The disclosed methods allow for detecting ctDNA or determining the tumor fraction from a biological sample from a patient that has, previously had, or is suspected of having cancer. As described in further detail below, the methods can be represented by two phases. In a first phase, or enrollment phase, a set of somatic mutations or variants that are specific to a patient are identified, and then filtered to generate a subset of somatic variants or mutations that include only specific types of somatic mutations or variants or show a preference for specific types of somatic mutations or variants. For the purposes of the disclosed methods, the subset of somatic mutations or variants will comprise or consist of multi-nucleotide variants, small indels, and genomic rearrangements for the reasons described herein. A panel of capture probes is then generated that are specific to the subset panel of somatic mutations or variants, which can be used to enrich a sample before sequencing.

Specific aspects of MRD processes are discussed in more detail below.

### III. Phase I-Signature Panel of Markers/Mutations and Capture Probes

a. DNA Library Preparation

In some embodiments of the methods disclosed herein, a DNA library is obtained or prepared from cfDNA obtained from a patient, e.g., a cancer patient. In some embodiments, a DNA library is obtained or prepared from the genome of the patient. In some embodiments, the DNA has been previously sequenced and mutations or variants identified.

When producing a DNA library from genomic DNA, the genomic DNA can be fragmented, for example by using a hydrodynamic shear or other mechanical force, or fragmented by chemical or enzymatic digestion, such as restriction digesting. This fragmentation process allows the DNA molecules present in the genome to be sufficiently short for analysis, such as sequencing or digital PCR cfDNA, however, is generally sufficiently short such that no fragmentation is necessary cfDNA originates from genomic DNA. A portion of the cfDNA obtained from a plasma sample of a cancer patient may originate from cancer cells (i.e., circulating tumor DNA or ctDNA) and a portion of the cfDNA may originate from non-cancer cells.

In some embodiments, the DNA molecules are subjected to additional modification, resulting in the attachment of oligonucleotides to the DNA molecules. The oligonucleotides can comprise an adapter sequence or a molecular barcode (or both). In some embodiments, the adapter sequence is common to all oligonucleotides in a plurality of oligonucleotides that are used to form the DNA library. In some embodiments, the molecular barcodes are unique or have low redundancy. By way of example, the oligonucleotide can be attached to the DNA molecules by ligation. Direct attachment of the oligonucleotides to the DNA molecules in the DNA library can be used, for example, when enrichment occurs in a downstream process. For example, in some embodiments, a DNA library is prepared by direct attachment of an oligonucleotide comprising a molecular barcode and an adapter sequence, followed by enrichment (for example, by hybridization) of DNA molecules comprising a region of interest or a portion of a region of interest.

In some embodiments, library preparation and enrichment occurs simultaneously. For example, in some embodiments, DNA molecules comprising a region of interest or a portion thereof are preferentially amplified. This can be done, for example, by combining the cfDNA (or genomic DNA), with oligonucleotides comprising a target-specific sequence, an adapter sequence, and a molecular barcode, and amplifying the DNA molecules. As before, in some embodiments, the adapter sequence is common to all oligonucleotides in a plurality of oligonucleotides, and the molecular barcode is unique or of low redundancy. The target-specific sequence is unique to the targeted region of interest or portion thereof. Thus, PCR amplification selectively amplifies the DNA molecules comprising the region of interest or portion thereof.

When the methods include the use of tags or molecular barcodes, the tag or molecular barcode may also be ligated to the fragments or included within the ligated adapter sequences. The independent attachment of the tag or molecular barcode, as opposed to incorporating the tag or molecular barcode, may vary with the enrichment method. For example, when using hybrid capture-based target enrichment the adapter can include the molecular barcode, when using PCR-targeted enrichment target-specific primer pairs and overhangs are used that will incorporate the sequencing adapters and sample-specific and molecular barcodes, and when using on-sequencer enrichment the adapter may be separately ligated from the tag or molecular barcode.

b. Panel of Mutations/Markers

In some embodiments, sequencing of the nucleic acid from the sample is performed using whole genome sequencing (WGS). In some embodiments, targeted sequencing is performed and may be either DNA or RNA sequencing. The targeted sequencing may be to a subset of the whole genome. In some embodiments the targeted sequencing is to introns, exons, intergenic regions, non-coding sequences or a combination thereof. In other embodiments, targeted whole exome sequencing (WES) of the DNA from the sample is performed. The DNA is sequenced using a next generation sequencing platform (NGS), which is massively parallel sequencing. NGS technologies provide high throughput sequence information, and provide digital quantitative information, in that each sequence read that aligns to the sequence of interest is countable. In certain embodiments, clonally amplified DNA templates or single DNA molecules are sequenced in a massively parallel fashion within a flow cell. In addition to high-throughput sequence information, NGS provides quantitative information, in that each sequence read is countable and represents an individual clonal DNA template or a single DNA molecule. The sequencing technologies of NGS include pyrosequencing, sequencing-by-synthesis with reversible dye terminators, sequencing by oligonucleotide probe ligation and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. Platforms for sequencing by synthesis are available from, e.g., Illumina, 454 Life Sciences, Helicos Biosciences, and Qiagen. Illumina platforms can include, e.g., Illumina's Solexa platform, Illumina's Genome Analyzer. Life Science platforms include, e.g., the GS Flex and GS Junior, and are described in U.S. Pat. No. 7,323,305. Platforms from Helicos Biosciences include the True Single Molecule Sequencing platform. Ion Torrent, an alternative NGS system, is available from ThermoScientific and is a semiconductor based technology that detects hydrogen ions that are released during polymerization of nucleic acids. Any detection method that allows for the detection of segregatable markers may be used with the assay provided for herein.

In some embodiments, whole genome sequencing (WGS) of the tumor and normal DNA is performed.

In other embodiments, Whole Exome Sequencing (WES) of the tumor and normal DNA is performed. WES comprises selecting DNA sequences that encode proteins, and sequencing that DNA using any high throughput DNA sequencing technology. Methods that can be used to target exome DNA include the use of polymerase chain reaction (PCR), molecular inversion probes (MIP), hybrid capture, and in-solution capture. The utility of targeted genome approaches is well established, and commercially available methods for WES include the Roche NimbleGen Capture Array (Roche NimbleGen Inc., Madison, WI), Agilent SureSelect (Agilent Technologies, Santa Clara, CA), and RainDance Technologies emulsion PCR (RainDance Technologies, Lexington, MA), IDT xGen® Exome Research Panel and others.

Sequence reads may comprise about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, about 500 bp, or more than 500 bp.

In some embodiments of the methods described herein, the somatic mutations identified will be analyzed and filtered to generate a subset panel of markers. For example, the subset panel of markers may comprise one or more types of somatic mutation, including but not limited to single-nucleotide variants (SNVs) multi-nucleotide variants, insertions and deletions (e.g., indel variants), and genomic rearrangements. In some embodiments, the subset panel will only include somatic mutations that comprise multiple changes compared to the normal sample, i.e., the subset panel will not include any SNVs. In some embodiments, the subset panel of somatic mutations can include greater than 50, up to 100, up to 200, up to 300, up to 400, up to 500, up to 600, up to 700, up to 800, up to 900, up to 1,000, up to 1,500, up to 2,000, up to 2,500, up to 3,000, up to 4,000, up to 5,000, up to 6,000, up to 7,000, up to 8,000, up to 9,000, up to 10,000, up to 11,000, up to 12,000, up to 13,000, up to 14,000, up to 15,000, or more than 15,000 mutations, which may comprise MNVs, small indels, genomic rearrangements, or combinations thereof. In other embodiments, the subset panel includes between 50 and 15,000 mutations, between 100 and 15,000 mutations, between 500 and 13,000 mutations, between 1,000 and 10,000 mutations, between 2,000 and 8,000 mutations, or between 4,000 and 6,000 mutations.

c. Capture Probes

The set of somatic variants or any subset therefrom (e.g., a “subset panel”) is represented by a set of oligonucleotide capture probes each designed to at least partially hybridize to a target sequence that has been identified to comprise a mutation or variant identified in the tumor sample from the patient or in the parental sequence. In some embodiments, the subset panel comprises capture probes comprising the subset of somatic mutations identified in the patient's tumor. In some embodiments, each capture probe is designed to selectively hybridize to a target sequence. The capture probe can be at least 70%, 75%, 80%, 90%, 95%, or more than 95% complementary to a target sequence. In some embodiments, the capture probe is 100% complementary to a target sequence. In some embodiments the capture probes are DNA probes. In other embodiments, the capture probes can be RNA.

The capture probe generally is sufficiently long to encompass the sequence of a somatic mutation, or corresponding normal sequence comprised in the genomic sequence targeted by the capture probe. The length and composition of a capture probe can depend on many factors including temperature of the annealing reaction, source and base composition of the oligonucleotide, and the estimated ratio of probe to genomic target sequence. Additionally, the length of the capture probe is dependent on the length of the target sequence it is designed to capture. The method provided utilizes cfDNA including circulating tumor DNA (ctDNA) as the source of the target sequences that are to be captured. Accordingly, as cfDNA is highly fragmented to an average of about 170 bp, the capture probe can be, for example, between 100 and 300 bp, between 150 and 250 bp, or between 175 and 200 bp. Currently, methods known in the art describe probes that are typically longer than 120 bases. In a current embodiment, if the allele is one or a few bases then the capture probes may be less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases, and this is sufficient to ensure equal enrichment from all alleles. When the mixture of DNA that is to be enriched using the hybrid capture technology is a mixture comprising cfDNA isolated from blood the average length of DNA is quite short, typically less than 200 bases. The use of shorter probes results in a greater chance that the hybrid capture probes will capture desired DNA fragments. Larger variations may require longer probes. For the purposes of the present disclosure, the variations of interest are more than one base in length. In some embodiments, targeted regions in the genome can be preferentially enriched using hybrid capture probes wherein the hybrid capture probes are shorter than 90 bases, and can be less than 80 bases, less than 70 bases, less than 60 bases, less than 50 bases, less than 40 bases, less than 30 bases, or less than 25 bases. In some embodiments, to increase the chance that the desired allele is sequenced, the length of the probe that is designed to hybridize to the regions flanking the polymorphic allele location can be decreased from above 90 bases, to about 80 bases, or to about 70 bases, or to about 60 bases, or to about 50 bases, or to about 40 bases, or to about 30 bases, or to about 25 bases.

Hybrid capture probes can be designed such that the region of the capture probe with DNA that is complementary to the DNA found in regions flanking the polymorphic allele is not immediately adjacent to the polymorphic site. Instead, the capture probe can be designed such that the region of the capture probe that is designed to hybridize to the DNA flanking the polymorphic site of the target is separated from the portion of the capture probe that will be in van der Waals contact with the polymorphic site by a small distance that is equivalent in length to one or a small number of bases. In an embodiment, the hybrid capture probe is designed to hybridize to a region that is flanking the polymorphic allele but does not cross it; this may be termed a flanking capture probe. The length of the flanking capture probe may be less than about 120 bases, less than about 110 bases, less than about 100 bases, less than about 90 bases, and can be less than about 80 bases, less than about 70 bases, less than about 60 bases, less than about 50 bases, less than about 40 bases, less than about 30 bases, or less than about 25 bases. The region of the genome that is targeted by the flanking capture probe may be separated by the polymorphic locus by 1, 2, 3, 4, 5, 6, 7, 8, 9, 10, 11-20, or more than 20 base pairs.

For small insertions or deletions, one or more probes that overlap the mutation may be sufficient to capture and sequence fragments comprising the mutation. Hybridization may be less efficient between the probe-limiting capture efficiency, typically designed to the reference genome sequence. To ensure capture of fragments comprising the mutation one could design two probes, one matching the normal allele and one matching the mutant allele. A longer probe may enhance hybridization. Multiple overlapping probes may enhance capture. Finally, placing a probe immediately adjacent to, but not overlapping, the mutation may permit relatively similar capture efficiency of the normal and mutant alleles.

For Short Tandem Repeats (STRs), a probe overlapping these highly variable sites is unlikely to capture the fragment well. To enhance capture a probe could be placed adjacent to, but not overlapping the variable site. The fragment could then be sequenced as normal to reveal the length and composition of the STR.

For large deletions, a series of overlapping probes, a common approach currently used in exon capture systems may work. However, with this approach it may be difficult to determine whether or not an individual is heterozygous. According to the method provided, custom probes are designed to ensure capture of the unique set of somatic mutations identified in the patient's tumor.

Capture probes can be modified to comprise purification moieties that serve to isolate the capture duplex from the unhybridized, untargeted cfDNA sequences by binding to a purification moiety binding partner. Suitable binding pairs for use in the invention include, but are not limited to, antigens/antibodies (for example, digoxigenin/antidigoxigenin, dinitrophenyl (DNP)/anti-DNP, dansyl-X-antidansyl, Fluorescein/anti-fluorescein, lucifer yellow/anti-lucifer yellow, and rhodamine anti-rhodamine); biotin/avidin (or biotin/streptavidin); calmodulin binding protein (CBP)/calmodulin; hormone/hormone receptor; lectin/carbohydrate; peptide/cell membrane receptor; protein A/antibody; hapten/antihapten; enzyme/cofactor; and enzyme/substrate. Other suitable binding pairs include polypeptides such as the FLAG-peptide (Hopp et al., BioTechnology, 6:1204-1210 (1988)); the KT3 epitope peptide (Martin et al., Science, 255:192-194 (1992)); tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)) and the antibodies each thereto. Further non-limiting examples of binding partners include agonists and antagonists for cell membrane receptors, toxins and venoms, viral epitopes, hormones such as steroids, hormone receptors, peptides, enzymes and other catalytic polypeptides, enzyme substrates, cofactors, drugs including small organic molecule drugs, opiates, opiate receptors, lectins, sugars, saccharides including polysaccharides, proteins, and antibodies including monoclonal antibodies and synthetic antibody fragments, cells, cell membranes and moieties therein including cell membrane receptors, and organelles. In some embodiments, the first binding partner is a reactive moiety, and the second binding partner is a reactive surface that reacts with the reactive moiety, such as described herein with respect to other aspects of the invention. In some embodiments, the oligonucleotide primers are attached to the solid surface prior to initiating the extension reaction. Methods for the addition of binding partners to capture oligonucleotide probes are known in the art, and include addition during (such as by using a modified nucleotide comprising the binding partner) or after synthesis. Additionally, the capture probes can be tethered to a solid surface, e.g., a magnetic bead, which facilitates the isolation of captured sequences.

### IV. Phase II-Detection and Monitoring Tumors by Analyzing cfDNA

a. Targeted Enrichment of a Region of Interest

The disclosed methods generally comprise enriching a target sequence in a region of interest. Examples of enrichment techniques include, but are not limited to, hybrid capture, selective circularization (also referred to as molecular inversion probes (MIP)), and PCR amplification of targeted regions of interest. Hybrid capture methods are based on the selective hybridization of the target genomic regions to user-designed oligonucleotides. The hybridization can be to oligonucleotides immobilized on high or low density microarrays (on-array capture), or solution-phase hybridization to oligonucleotides modified with a ligand (e.g., biotin) which can subsequently be immobilized to a solid surface, such as a bead (in-solution capture). Molecular inversion probe (MIP)-based method relies on construction of numerous single-stranded linear oligonucleotide probes, consisting of a common linker flanked by target-specific sequences. Upon annealing to a target sequence, the probe gap region is filled via polymerization and ligation, resulting in a circularized probe. The circularized probes are then released and amplified using primers directed at the common linker region. PCR-based methods employ highly parallel PCR amplification, where each target sequence in the sample has a corresponding pair of unique, sequence-specific primers. In some embodiments, enrichment of a target sequence occurs at the time of sequencing.

In the second phase of the method, samples that are used for determining the tumor fraction of the patient include samples that contain nucleic acids that are cell-free. Cell-free nucleic acids, including cfDNA, can be obtained by various methods from biological samples including but not limited to plasma, serum, and urine. Other biological fluid samples include, but are not limited to blood, sweat, tears, sputum, ear flow, lymph, saliva, cerebrospinal fluid, ravages, bone marrow suspension, vaginal flow, transcervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures, e.g., blood, plasma, serum, sweat, tears, sputum, urine, ear flow, saliva or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample.

In various embodiments the cfDNA present in the sample can be enriched specifically or non-specifically prior to use (e.g., prior to capture and sequencing). Non-specific enrichment of sample DNA refers to the whole genome amplification of the DNA fragments of the sample that can be used to increase the level of the sample DNA prior to capture and sequencing. Non-specific enrichment can be the selective enrichment of exomes. Methods for whole genome amplification are known in the art. Degenerate oligonucleotide-primed PCR (DOP), primer extension PCR technique (PEP) and multiple displacement amplification (MDA) are examples of whole genome amplification methods. In some embodiments, the sample is unenriched for cfDNA.

As is described elsewhere herein, cfDNA is present as fragments averaging about 170 bp. Accordingly, further fragmentation of cfDNA is not needed. In some embodiments, sufficient cfDNA is obtained from a 10 ml blood sample to confidently determine the presence or absence of cancer in a patient. The blood samples used in the method provided can be of about 5 ml, about 10 ml, about 15 ml, about 20 ml, about 25 ml or more than 25 ml. Typically, 20 ml of blood plasma contains between 5,000 and 10,000 genome equivalents, and provides more than sufficient cfDNA for determining tumor fraction according to the method provided. In some embodiments, sufficient cfDNA is obtained from 10 ml to 20 ml of blood to determine tumor fraction.

To separate cfDNA from cells in a sample, various methods including, but not limited to fractionation, centrifugation (e.g., density gradient centrifugation), DNA-specific precipitation, or high-throughput cell sorting and/or other separation methods can be used. Commercially available kits for manual and automated separation of cfDNA are available (Roche Diagnostics, Indianapolis, Ind., Qiagen, Germantown, MD).

cfDNA can be end-repaired, and optionally dA tailed, and double-stranded adaptors comprising sequences complementary to amplification and sequencing primers are ligated to the ends of the cfDNA molecules to enable NGS sequencing, e.g., using an Illumina platform. Additionally, each of the double-stranded adaptors further comprises a non-random barcode sequence, which serves to differentiate individual cfDNA molecules. In some embodiments, the barcode sequences are random sequences. In other embodiments, the barcode sequences are non-random barcode sequences. Non-random barcode sequences provide a significant advantage over random barcode sequences because non-random barcode sequences enable unambiguous identification of the sequencing reads described below. The nonrandom barcode sequences are designed specifically to be base-balance both within and across all barcodes. Additionally, in some embodiments, the nonrandom barcodes can comprise a T nucleotide at 3′ end, which is complementary to the A nucleotide of dA-tailed cfDNA molecules. In embodiments utilizing a T nucleotide overhang at 3′ end of the barcode, barcodes of three different lengths can be designed to avoid a single base flashing across the entire flowcell of the sequencer. Nonrandom barcode sequences can be present in adaptors as sequences of 13, 14, and 15 bp; 10, 11, and 12 bp; 11, 12, and 13 bp; 13, 14, and 15 bp; 14, 15, and 16 bp; 15, 16, and 17 bp, and the like. In some embodiments, the shortest barcode sequence can be 8 bp and the longest barcode sequence can be 100 bp.

Each sequence of the set of somatic variants or subset of somatic variants (“subpanel”) that is present in the cfDNA sample is targeted by one or more capture probes described elsewhere herein, and is isolated for further analysis.

b. Sequencing and Analysis

The disclosed methods generally comprise sequencing one or more samples. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent sequencing), nanopore sequencing, pyrosequencing (454), sequencing by synthesis, sequencing by ligation (SOLID sequencing), sequencing by electron microscopy, dideoxy sequencing reactions (Sanger method), massively parallel sequencing, polony sequencing, duplex sequencing, and DNA nanoball sequencing. In some embodiments, sequencing involves hybridizing a primer to the template to form a template/primer duplex, contacting the duplex with a polymerase enzyme in the presence of a detectably labeled nucleotides under conditions that permit the polymerase to add nucleotides to the primer in a template-dependent manner, detecting a signal from the incorporated labeled nucleotide, and sequentially repeating the contacting and detecting steps at least once, wherein sequential detection of incorporated labeled nucleotide determines the sequence of the nucleic acid. In some embodiments, the sequencing comprises obtaining paired end reads. The accuracy or average accuracy of the sequence information may be greater than 80%, 90%, 95%, 99% or 99.98%. In some embodiments, the sequence information obtained is more than 50 bp, 100 bp or 200 bp. The sequence information may be obtained in less than 1 month, 2 weeks, 1 week 1 day, 3 hours, 1 hour, 30 minutes, 10 minutes, or 5 minutes. The sequence accuracy or average accuracy may be greater than 95% or 99%. Examples of detectable labels include radiolabels, florescent labels, enzymatic labels, etc. In some embodiments, the detectable label may be an optically detectable label, such as a fluorescent label. Examples of fluorescent labels include cyanine, rhodamine, fluorescien, coumarin, BODIPY, alexa, or conjugated multi-dyes. In some embodiments, the nucleotide is flagged if one or more of its sequence segments are substantially similar to one or more sequence segments of another nucleotide within the same partition.

Some methods of sequencing may require or involve a prior target enrichment step. For example, use of on-sequencer enrichment, such as with a nanopore sequencer, allows for the simultaneous enrichment and sequencing of the sequence library by real-time rejection of molecules that are not from the region of interest. Alternatively, sequences can be selectively and preferentially sequenced from the region of interest.

Captured sequences can be analyzed using the sequencing-by-synthesis technology of Illumina, which uses fluorescent reversible terminator deoxyribonucleotides. The reads generated by the sequencing process are aligned to a reference sequence and associated with a sequence of the somatic sequence panel specific for the patient. Mapping of the sequence reads can be achieved by comparing the sequence of the reads with the sequence of the reference genome to determine the specific genetic information, and optionally the chromosomal origin of the sequenced nucleic acid (e.g., cfDNA) molecule. A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al, Genome Biology 10: R25.1-R25.10 [2009]), or ELAND (Illumina, Inc., San Diego, Calif., USA). In one embodiment, the sequencing data is processed by bioinformatic alignment analysis for the Illumina Genome Analyzer, which uses the Efficient Large-Scale Alignment of Nucleotide Databases (ELAND) software. Additional software includes SAMtools (SAMtools, Bioinformatics, 2009, 25 (16): 2078-9), and the Burroughs-Wheeler block sorting compression procedure which involves block sorting or preprocessing to make compression more efficient.

The barcoded cfDNA fragments isolated from the patient's fluid sample, e.g., blood sample, can be amplified, e.g., by PCR, and captured using the hybrid probes. Capturing of the barcoded fragments comprises obtaining single strands of barcoded cfDNA, and hybridizing the barcoded cfDNA with different hybrid probes. Each of the different hybrid probes hybridizes to a single-stranded barcoded cfDNA target sequence to form a target-hybrid probe duplex. The duplex is isolated from unhybridized cfDNA by binding the purification binding moiety comprised in the hybrid probe to the corresponding purification moiety binding partner. As described elsewhere herein, the corresponding purification moiety binding partner can be immobilized on a solid surface, e.g., a magnetic bead, which facilitates the separation of the capture duplex from unhybridized cfDNA molecules in solution. The barcoded cfDNA of the duplex is released, and is subjected to sequencing using an NGS instrument.

The error rate in sequencing using NGS methods is of approximately 1 in 500 bases which results in many sequencing errors. The high error rate becomes problematic especially when attempting to identify somatic mutations in mixtures of DNA sequences comprising only a small fraction of mutated species or sequences comprising single nucleotide variants. The methods described herein avoid such errors by analyzing target sequences that comprise somatic mutations having multiple changes relative to a reference sequence. Additionally, NGS methods typically utilize single stranded DNA as the primary source of sequencing material. Any error included during the amplification step of the DNA molecule prior to sequencing is perpetuated, and becomes indistinguishable as an extraneous technology-dependent mistake. Chemical errors occur at a frequency of approximately in 1000 bases. The combination of sequencing and chemical errors obscure the limit of detection (LOD).

Accordingly, in some embodiments, double-stranded sequencing of the cfDNA is performed. As described elsewhere herein cfDNA can be end-repaired, and optionally dA tailed, and double-stranded adaptors comprising sequences complementary to amplification and sequencing primers are ligated to the ends of the cfDNA molecules to enable NGS sequencing, e.g., using an Illumina platform.

The tumor fraction can then be calculated as the proportion of different cfDNA sequences each comprising at least one somatic mutation, i.e., ctDNA sequences, relative to the total number of different cfDNA, i.e., ctDNA and corresponding normal sequences. Unlike the single-stranded approach, the current method corrects for random sequencing errors. Alternatively, the tumor fraction can be determined by fitting a binomial mixture model of variant counts and total counts across the entire panel of variants assayed, where the mixture components are the tumor, non-tumor, or germline variants and the weighting of each class is determined by the probability that a variant belongs to that class. Enumeration of mutated and unmutated allelic sequences can be accomplished by analyzing the countable sequence reads obtained from the sequencing process.

c. Molecular Barcodes

In some embodiments, an identifier sequence, i.e., a molecular barcode, may be used to identify unique DNA molecules or target sequences in a DNA library. Molecular barcodes aid in reconstruction of a contiguous DNA sequences or assist in copy number variation determination. Exemplary markers include nucleic acid binding proteins, optical labels, nucleotide analogs, nucleic acid sequences, and others known in the art.

In some embodiments, the molecular barcode is a nanostructure barcode. In some embodiments, the molecular barcode comprises a nucleic acid sequence that when joined to a target polynucleotide serves as an identifier of the sample or sequence from which the target polynucleotide was derived. In some embodiments, molecular barcodes are at least 3, 4, 5, 6, 7, 8, 9, 10, 11, 12, 13, 14, 15, or more nucleotides in length. In some embodiments, molecular barcodes are shorter than 10, 9, 8, 7, 6, 5, or 4 nucleotides in length. In some embodiments, each molecular barcode in a plurality of molecular barcodes differ from every other molecular barcode in the plurality at least three nucleotide positions, such as at least 3, 4, 5, 6, 7, 8, 9, 10, or more positions. In some embodiments, molecular barcodes associated with some polynucleotides are of different length than molecular barcodes associated with other polynucleotides. In general, molecular barcodes are of sufficient length and comprise sequences that are sufficiently different to allow the identification of samples based on molecular barcodes with which they are associated. In some embodiments, both the forward and reverse adapter comprise at least one of a plurality of molecular barcode sequences. In some embodiments, each reverse adapter comprises at least one of a plurality of molecular barcode sequences, wherein each molecular barcode sequence of the plurality of molecular barcode sequences differs from every other molecular barcode sequence in the plurality of molecular barcode sequences.

In some embodiments, every molecular barcode in a set is unique, that is, any two molecular barcodes chosen out of a given set will differ in at least one nucleotide position. Furthermore, it is contemplated that molecular barcodes have certain biochemical properties that are selected based on how the set will be used. For example, certain sets of molecular barcodes that are used in an RT-PCR reaction should not have complementary sequences to any sequence in the genome of a certain organism or set of organisms. A requirement for non-complementarity helps to ensure that the use of a particular molecular barcode sequence will not result in mis-priming during molecular biological manipulations requiring primers, such as reverse transcription or PCR. Certain sets satisfy other biochemical properties imposed by the requirements associated with the processing of the sequence molecules into which the barcodes are incorporated.

Examples of sequencing technologies for sequencing molecular barcodes, as well as any generated nucleotide-based sequence, include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent sequencing), nanopore sequencing, pyrosequencing (454), sequencing by synthesis, sequencing by ligation (SOUD sequencing), sequencing by electron microscopy, dideoxy sequencing reactions (Sanger method), massively parallel sequencing, polony sequencing, and DNA nanoball sequencing.

In some embodiments, molecular barcodes are used to improve the power of copy-number calling algorithms by reducing non-independence from PCR duplication. In another embodiment, molecular barcodes can be used to improve test specificity by reducing sequence error generated during amplification.

### V. Improvements to MRD Using Allele Frequency and Machine Learning

Although MRD methods generally provide significant clinical utility in tracking treatment, recurrence, and prognosis of cancer patients, certain aspects of prior MRD processes can be improved with the disclosed methods. Specifically, MRD requires tumor/cancer profiling to identify somatic variants present in a tumor of interest. The process of tumor profiling typically yields an excess of variant sites, and therefore a subset of those sites can be selected to form the tumor/patient-specific panel (i.e., a signature panel or a subset of somatic variants) for target enrichment and sequencing from sample-derived (e.g., plasma-derived) cell-free DNA (cfDNA). The present disclosure is the first to recognize that the composition of the selected sites is a determinant of the sensitivity and specificity of cfDNA and that there is a higher probability of detecting somatic variants derived from ctDNA when the expected variant allele frequency is maximized.

The disclosed methods prioritize sites to generate a subset of somatic variants based on a plurality of features such as using a relationship between copy number and allele balance such that tumor somatic variants are overrepresented in cfDNA. Machine learning models trained with genomic data (e.g., DNA sequencing data, whole genome sequencing data, whole exome sequencing data, targeted genomic sequencing data, cfDNA sequencing data, chromatin immunoprecipitation sequencing data, reference genome data, transcriptomics data, epigenomics data, or proteomics data) are also utilized to select the subset of somatic variants.

Accordingly, the disclosed methods related to improving the detection, monitoring, and treatment of a cancer patient undergoing MRD assessment. The patient can be suspected or known to harbor a solid tumor, or the patient may have previously harbored a solid tumor. In some aspects the solid tumor is a tumor of a tissue or organ. In other aspects, the solid tumor is a metastatic mass of a blood borne cancer. The present method can also be applicable to the detection and/or monitoring of blood borne or hematological cancers, such as leukemia or lymphoma.

The disclosed methods are also applicable to MRD testing, wherein the patient has previously been treated for a cancer, and may be considered in remission, however a small number of cancer cells remain in the body. The number of remaining cells may be so small that they do not cause any physical signs or symptoms and often cannot even be detected through traditional methods, such as viewing cells under a microscope and/or by tracking abnormal serum proteins in the blood. An MRD positive test results means that residual (remaining) disease was detected. A negative result means that residual disease was not detected. MRD testing may be used to measure the effectiveness of treatment and to predict if a patient is at risk of relapse. When a patient tests positive for MRD, it means that there are still residual cancer cells in the body after treatment. When MRD is detected, this is known as “MRD positivity.” When a patient tests negative, no residual cancer cells were found. When no MRD is detected, this is known as “MRD negativity.”

For the purposes of the disclosed methods, the variants that are targeted for enrichment are generally somatic variants; however, the variants may also include de novo genetic variant. That is, if the genetic variant is not present in non-cancerous cells of the cancer patient, and the described method indicates that the genetic variant is distinguishable from the cancer patient genome, then the genetic variant is a de novo variant. Accordingly, some embodiments of the disclosed methods may comprise determining whether a genetic variant is an inherited genetic variant or a de novo genetic variant.

In a second phase, monitoring of the status of the cancer in the patient is performed using the patient's panel of probes (e.g., capture probes) to identify somatic mutations that are circulating as cfDNA. The second phase is non-invasive and requires clinically viable amounts of a biological fluid, e.g., a peripheral blood draw of about 5-25 ml (e.g., about 5, about 10, about 15, about 20, or about 25 mls), which can be repeated as frequently as desired to detect changes in the patient's cancer. A clinically viable amount of biological fluid, e.g., whole blood, typically comprises at least 1000 genome equivalents, at least 2000 genome equivalents, at least 3000 genome equivalents, at least 4000 genome equivalents, at least 5000 genome equivalents, at least 6000 genome equivalents, at least 7000 genome equivalents, at least 8000 genome equivalents, at least 9000 genome equivalents, at least 10000 genome equivalents, at least 11000 genome equivalents, at least 12000 genome equivalents, or at least 15000 genome equivalents. In some embodiments, the second phase of the method utilizes a whole blood sample of between 5 ml and 20 ml, comprising between 3000 and 15000 genome equivalents.

a. Expected Variant Allele Frequency

The expected variant allele frequency is a function of the fraction of ctDNA present in cfDNA, the number of variant allele copies in the tumor sample and the ratio of the variant allele to reference allele (e.g., allele balance) in the tumor sample. This approach is modeled on the relationship between the probability of observing alternative allele counts, p, at a site given allele balance in the tumor (ABT) copy number of the site in both tumor (CNT) and normal (CNx) and the fraction of tumor DNA in the sample (TF), where c stands for the error rate:

$$p = \frac{TF \cdot {CN}_{T}}{TF \cdot {CN}_{T} + ( 1 - TF ) {CN}_{N}} \cdot {AB}_{T} + c$$

Each variant will be ranked by an expected p at 0.005% TF using allele balance and copy number in the tumor sample, assuming copy number in the non-tumor sample is 2. This approach accounts for the sub-clonal variants on detection sensitivity.
b. Machine Learning Model

A panel of sequences comprising a set of somatic variants specific to the tumor of a patient can be identified as follows. DNA (e.g., cfDNA or genomic DNA) is isolated from the tumor and from a non-tumor sample, such as normal tissue (i.e., non-cancerous tissue) or fluid sample obtained from a cancer patient, and sequenced. DNA sequences from the tumor and non-tumor samples are compared, and a set of somatic variants specific to the patient's cancer are identified. The set of somatic variants are based in the differences between the sequences of DNA from the tumor sample and sequences of DNA from the non-tumor sample. In some embodiments, data associated with a tumor sample and a non-tumor sample from a cancer patient is retrieved by a processor. In some embodiments, the data is sequenced DNA (e.g., cfDNA or genomic DNA). In some embodiments, the set of somatic variants based on differences between sequences of DNA from the tumor sample and sequences of DNA from the non-tumor sample are generated by a computer device. In some embodiments, a training data set is generated by a processor comprising sequenced DNA from the tumor sample and the non-tumor sample, and a set of somatic variants based on the differences between sequences of DNA from the tumor sample and the non-tumor sample. As described herein, the training dataset can be used to train one or more machine learning models, such that when trained the machine learning model can ingest a set of mutations for a new patient and select a subset of the mutations that are suitable for tracking a patient's progress over the course of treatment or cancer recurrence.

The panel (e.g., set of somatic variants) is then filtered to generate a subset of somatic variants based on the expected variant allele frequency and/or based on a plurality of features selected by a machine learning model trained with genomic data. In some embodiments, the genomic data comprises DNA sequencing data, whole genome sequencing data, whole exome sequencing data, targeted genomic sequencing data, cfDNA sequencing data, chromatin immunoprecipitation sequencing data, reference genome data, transcriptomics data, epigenomics data, or proteomics data. In some embodiments, the expected variant allele frequency based on copy number and allele balance of each of the somatic variants in the set of somatic variants is calculated using a computer processor. The subset of somatic variants are then selected based on a reference threshold. In some embodiments, selecting a subset of somatic variants comprises subtracting an expected error rate from the expected variant allele frequency.

The selection process may be performed automatically using a processor implementing an algorithm. The algorithm may be configured to analyze the data and perform various filtering protocols using the reference threshold. In some embodiments, the reference threshold may be a static threshold that can be set by a system administrator or any other party with access to the algorithm implementing the threshold. Additionally or alternatively, the reference threshold may be a dynamic threshold that is generated by the algorithm (or using a secondary algorithm or using a set of rules). For instance, another protocol may be implemented, such that the data is analyzed, and the reference threshold is uniquely calculated for the set of data being analyzed. Moreover, whether to use a static or dynamic reference threshold may also be determined using one or more triggering conditions. Alternatively, the selection process may comprise ranking somatic variants and then selecting suitable variants from among the ranked list.

In some embodiments, the subset of somatic variants are above a reference threshold. In some embodiments, the set of somatic variants are filtered based on plurality of features selected by machine learning models to generate a subset of somatic variants. In some embodiments, the plurality of features are selected by a machine learning model trained with genomic data, such as DNA sequencing data, whole genome sequencing data, whole exome sequencing data, targeted genomic sequencing data, cfDNA sequencing data, chromatin immunoprecipitation sequencing data, reference genome data, transcriptomics data, epigenomics data, or proteomics data. In some embodiments, and output is provided indicating the subset of somatic variants that are suitable for isolating ctDNA. In some embodiments, a processor is training a machine learning model using a training dataset to ingest data associated with a second tumor sample and a second non-tumor sample from a patient and predict a subset of somatic variants.

The set of somatic variants is filtered based on the expected variant allele frequency above a reference threshold alone or in combination with a plurality of features selected by machine learning models to generate a subset of somatic variants. Alternatively, the set of somatic variants can be filtered to generate a subset of somatic variants based on a plurality of features selected by machine learning models. In some embodiments, using a first computer model an allele frequency of each somatic variant can be calculated by a processor and using a second computer model, at least one valid target site associated with at least one somatic variant can be calculated by the processor. The processor can further select the subset of variants in accordance with an output of the first computer model and the second computer model.

The subset of somatic variants serves as a signature panel for the patient that can be sequenced at various stages of the disease, i.e., the signature panel can be screened to determine the presence of cancer at surgery following diagnosis; during cancer treatment, e.g., at intervals during chemotherapy or radiation therapy, to monitor the efficacy of the treatment; at intervals during remission to confirm continued absence of disease; and/or to detect recurrence of the disease. The composition of the selected somatic variants for the subset is a key determinant for the sensitivity and specificity of the methods described herein.

Next, a set of probes is obtained (e.g., capture probes). The set of capture probes comprises sequences that are capable of hybridizing to specific target sequences in the patient's genome and that encompass the sites comprising the subset of somatic variants identified in the tumor tissue. More particularly, the set of probes can hybridize to target sequences comprising the subset somatic variants.

Subsequently, the presence of ctDNA and/or the tumor fraction in a fluid sample from the same patient is determined. Determining the tumor fraction comprises obtaining cfDNA from the patient, and using the capture probes designed for the patient-specific subset panel to capture cfDNA target sequences comprising tumor sequences (i.e., ctDNA). The captured DNA is sequenced, and the sequences can be analyzed and enumerated. The tumor fraction can be determined by fitting a binomial mixture model of variant counts and total counts across the entire panel of variants assayed, where the mixture components are the tumor, non-tumor, or germline variants and the weighting of each class is determined by the probability that a variant belongs to that class. Enumeration of mutated and unmutated allelic sequences can be accomplished by analyzing the countable sequence reads obtained from the sequencing process. The method does not necessitate that all somatic mutations in the patient's signature panel be detected. Rather, a test or assay can be considered positive (i.e., ctDNA is present) if as little as a single somatic mutation in the patient's signature panel is detected.

Various actions discussed herein, such as methods and processes discussed herein may be performed (at least partially) by one or more processors implementing/utilizing one or more machine learning models.

The Figure illustrates an example computer environment 100 that can be used to provide a network-based implementation of the methods and processes described herein. Specifically, The Figure illustrates components of a system 100 for a mutation analysis system, according to an embodiment. The system 100 may include an analytics server 110a, system database 110b, a machine learning model 111, electronic data sources 120a-d (collectively electronic data sources 120), end-user devices 140a-c (collectively end-user devices 140), and an administrator computing device 150.

The system 100 is not confined to the components described herein and may include additional or other components not shown for brevity, which are to be considered within the scope of the embodiments described herein.

The above-mentioned components may be connected to each other through a network 130. Examples of the network 130 may include, but are not limited to, private or public local-area-networks (LAN), wireless LAN (WLAN) networks, metropolitan area networks (MAN), wide-area networks (WAN), and the Internet. The network 130 may include wired and/or wireless communications according to one or more standards and/or via one or more transport media. Communication over the network 130 may be performed in accordance with various communication protocols such as Transmission Control Protocol and Internet Protocol (TCP/IP), User Datagram Protocol (UDP), and Institute of Electrical and Electronics Engineers (IEEE) communication protocols. In one example, the network 130 may include wireless communications according to Bluetooth specification sets or another standard or proprietary wireless communication protocol. In another example, the network 130 may also include communications over a cellular network, including, e.g., GSM (Global System for Mobile Communications), CDMA (Code Division Multiple Access), or EDGE (Enhanced Data for Global Evolution) networks.

The analytics server 110a may generate and display an electronic platform configured to receive information and output results of execution of the machine learning model 111. The electronic platform may include a graphical user interface (GUI) displayed on the electronic data sources 120, the end-user devices 140, and/or the administrator computing device 150. An example of the electronic platform generated and hosted by the analytics server 110a may be a web-based application or a website configured to be displayed on various electronic devices, such as mobile devices, tablets, personal computers, and the like. Simply put, the analytics server 110a may implement the platform to receive data and instructions from end users (using the end-user devices 140); The analytics server 110a may then execute the machine learning model 111 accordingly and display the results (e.g., a list of “suitable” or “best” mutations for tracking a patient's progress over the course of treatment or cancer recurrence) on the platform.

The analytics server 110a may be any computing device comprising a processor and non-transitory, machine-readable storage capable of executing the various tasks and processes described herein. The analytics server 110a may employ various processors such as a central processing unit (CPU) and graphics processing unit (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

The electronic data sources 120 may represent various sources that contain, retrieve, and/or access data needed to train the machine learning model 111. For instance, the analytics server 110a may use a laboratory computer 120a, medical professional device 120b, server 120c (associated with a laboratory), and/or database 120d (associated with a research lab, a clinic, and/or any third party providing data) to retrieve and receive data. As used herein, the electronic data sources may include any electronic source containing data (e.g., WGS data) that can be used to generate a training dataset in order to (ultimately) train the machine learning model 111. Even though referred to herein as “laboratory” devices, these devices may not always be operated in laboratories. Therefore, no limitation is intended by this term.

When generating the training data, the analytics server 110a may execute various algorithms to translate raw data received or retrieved from the electronic data sources 120 into machine-readable objects that can be stored and processed by other analytical processes as described herein.

End-user devices 140 may be any computing device comprising a processor and a non-transitory, machine-readable storage medium capable of performing the various tasks and processes described herein. Non-limiting examples of an end-user device 140 may be a workstation computer, laptop computer, tablet computer, or server computer. During operation, various users may use end-user devices 140 to access the GUI operationally managed by the analytics server 110a. Specifically, the end-user devices 140 may include laboratory computer 140a, laboratory server 140b, and a user device 140c. Even though referred to herein as “end-user” or “laboratory” devices, these devices may not always be operated by end-users or in laboratories. Therefore, no limitation is intended by these terms.

The administrator computing device 150 may represent a computing device operated by a system administrator. The administrator computing device 150 may be configured to display various attributes and predictions generated by the analytics server 110a (e.g., various analytic metrics determined during training of one or more machine learning models and/or systems); monitor various models 111 utilized by the analytics server 110a, electronic data sources 120, and/or end-user devices 140; review feedback; and/or facilitate training or retraining (calibration) of the machine learning model 111 that are maintained by the analytics server 110a.

The machine learning model 111 may be stored in the system database 110b. The machine learning model 111 may be trained using data received or retrieved from the electronic data sources 120 and may be executed using data received from the end-user devices 140. In some embodiments, the machine learning model 111 may reside within a data repository that is local or specific to a laboratory or an end user (e.g., client). In other embodiments, the machine learning model may be stored centrally where access to its predictions are controlled per client basis.

It should be understood that any alternative and/or additional machine learning model(s) may be used to implement similar learning engines. As described herein, the analytics server 110a may store the machine learning model 111 (e.g., neural networks, random forest, support vector machines, regression models, recurrent models, etc.) in an accessible data repository. The analytics server 110a may retrieve the machine learning model 111 and train it to predict a suitable subset of mutations for tracking a patient's progress over the course of treatment or cancer recurrence. Various machine learning techniques can be used to train the machine learning model 111, such as supervised learning techniques, unsupervised learning techniques, or semi-supervised learning techniques, among others.

In operation, the analytics server may train the machine learning model 111, such that (at the inference phase), the machine learning model 111 may determine one or more mutations from a set of available mutations. Using a higher number of target sites may increase the number of opportunities to detect ctDNA. Thus, if the machine learning model 111 chooses a fixed number of target sites for the assay, there may be a difference in the chance of observing ctDNA (e.g., if the accuracy in identifying target sites is 100% versus 90%). Using the methods and systems discussed herein, the analytics server can use one or more processors discussed herein to improve the accuracy of target site identification to increase the average number of valid target sites included in the MRD panels. The analytics server 110a may use the machine learning model 111 and/or an algorithmic approach in order to achieve this.

The predicted mutations could be used alone, or integrated with CN and allele balance to boost the probability of observing an ALT allele. Integration could also include serial ranking methods (e.g., ranking by the probability that the target site is valid, then by the probability of observing an ALT count) or by complete probabilistic modeling techniques (e.g., where the probability of observing an ALT count is adjusted by the probability that the target site is valid).

In operation, the analytics server 110a may first retrieve data from the electronic data sources 120. Accordingly, WGS data collection and somatic variant calling can be performed on tumor/normal pairs for a defined number of data points (e.g., 40). Subsequently, target sites can be selected based on somatic calling results. In some embodiments, sites can be selected based on the high confidence of the calls. Additionally or alternatively, the case sites can be selected, such that confidence and other sequencing metrics can span the parameter space.

After the selection of sites, probes can be ordered to cover the selected sites. The analytics server 110a may then selectively enrich the target sites in the tumor and normal samples using the probes in order to generate sequence libraries containing unique molecular identifiers (UMIs) and sequenced at high depth. In some embodiments, the analytics server 110a can receive an indication of the UMIs from a lab employee or any other sources.

Sequenced data can then be de-duplicated using various protocols (e.g., read start and stop positions and UMIs, which reduces sequencing noise, yielding cleaner data). Subsequently, targeted sites can be classified as True Positive (TP) or False Positive (FP) based on the frequency of the ALT allele observed in the sample pairs. In this way, the data (that is now no longer raw because it has been denoised and de-duplicated) can be labeled, such that training the machine learning model 111 can be achieved in a supervised or semi-supervised manner.

In order to label the data, the analytics server 110a may use various defined rules and protocols. For instance, one non-limiting rule may dictate that to be a TP, the ALT allele frequency must be higher in the tumor than the normal sample AND the ALT frequency must be at least a minimum frequency AND the normal frequency must be less than a maximum frequency. The analytics server 110a may use any other defined rule that uses thresholding (whether dynamic or static) to label the data.

The analytics server 110a may also extract a feature set for all (or some) of the target sites. Features, as used herein, may be based on raw sequencing outcomes (e.g., allele counts or strand bias), summary of sequence outcomes (e.g., mean base quality for REF or ALT alleles), metrics derived from sequencing outcomes of the tumor and normal pair (e.g., ratios of tumor and normal sequence metrics), outputs of somatic variant calling algorithms (e.g., true/false, confidence or variant score), and/or sample-level metrics (e.g., tumor mutation burden or cancer type), or sequence context (e.g., mutation type, GC content, local complexity). In some embodiments, the most predictive feature can correspond to a fisher log (p-value) for a 2×2 contingency table of REF and ALT counts of the tumor and normal.

Using the training data (as generated using the methods and protocols discussed herein), the machine learning model 111 can be trained. In some embodiments, the machine learning model 111 may refer to (or utilize) a random forest model with features and variant classifications. In some embodiments, the training data can be segmented into training and validation/testing folds. For instance, the data can be folded into a 60/40 split (60% training/40% test). During training, various hyperparameter tuning protocols can be implemented to select the optimal number of trees and the number of variables in each tree (of the random forest). These hyperparameters can then be used to train the machine learning model 111.

The analytics server may use various methodologies to evaluate the performance of the machine learning model 111. For instance, in some embodiments, out-of-bag error to assess the performance rather than k-fold cross-validation; however, other implementations and embodiments may include other methodologies.

### VI. EXAMPLES

The present invention is described in further detail in the following examples which are not in any way intended to limit the scope of the invention as claimed. All references cited are herein specifically incorporated by reference for all that is described therein. The following examples are offered to illustrate, but not to limit the claimed invention.

### Example 1: Selecting a Patient Specific Subset Panel of Somatic Variants and Diagnosing MRD

Since somatic calling from WGS typically yields an excess of variant sites, a subset of those sites is selected to form a tumor/patient-specific panel (e.g., subset of somatic variants) for target enrichment and sequencing from plasma-derived cfDNA. The composition of the selected sites is a key determinant for the sensitivity and specificity of the cfDNA assay. Sites are prioritized based on expected variant allele frequency and/or machine learning

Preparing subset panel: DNA is extracted from a normal and tumor sample from a patient and a sequencing library is prepared for each sample. The samples are sequenced by whole genome sequencing and somatic variants identified. A subpanel of the somatic variants is then selected by selecting sites having an expected variant allele frequency based on copy number and allele balance reference cut off and/or a plurality of features selected by a machine learning model trained with genomic data, such as, DNA sequencing data, whole genome sequencing data, whole exome sequencing data, targeted genomic sequencing data, cfDNA sequencing data, chromatin immunoprecipitation sequencing data, reference genome data, transcriptomics data, epigenomics data, or proteomics data. Hybrid capture probes are then generated for the somatic variants of the subpanel.

Diagnosing MRD: cfDNA is extracted from the patient and selectively enriched using hybrid capture probes for the somatic variants of the subpanel. The enriched library is sequenced to generate sequencing reads for each of the somatic variants of the subset panel. MRD is diagnosed based on the presence of patient/tumor specific somatic mutation in the cfDNA sample.

### Example 2: Minimizing Error in Target Selection by Determining a Positive Predictive Value (PPV)

Whole genome sequencing was performed on 11 clinical sample sets of a formalin fixed paraffin embedded (FFPE) tumor sample and FFPE normal sample per set and two cell line sample sets of a tumor cell line and a normal cell line. Somatic variants were called with an ensemble of somatic variant callers. A panel of targets were selected for each sample based on somatic variant called and the panel was constructed.

Each panel was used for hybrid capture and subsequent sequencing of its match tumor and normal sample was performed. Allele counts at each target were collected. Targets for which the alternate allele was detected in the tumor sample and not the normal sample were classified as true somatic variants. Targets for which the alternate allele was not detected in the tumor sample or was detected in the normal sample were classified as false somatic variants.

Data was collected for each target in each sample, including calling outputs (i.e., MuSE Tier, LoFreq score, Strelka score and Mutect2 call), raw allele counts (i.e., total count, REF count, and ALT counts for tumor and normal, ALT bias for tumor, and REF bias for normal), aggregated count data (i.e., mean mapping quality, base quality and base position for ref and alt alleles in the tumor), metrics derived from count data (i.e., ratios of counts or means, fisher p-values for counts) and sequence features (i.e., mutation type, GC content, sequence entropy and local sequence context). A random forest (RF) classifier was trained using the collected data and variant classifications derived from the hybrid capture sequencing.

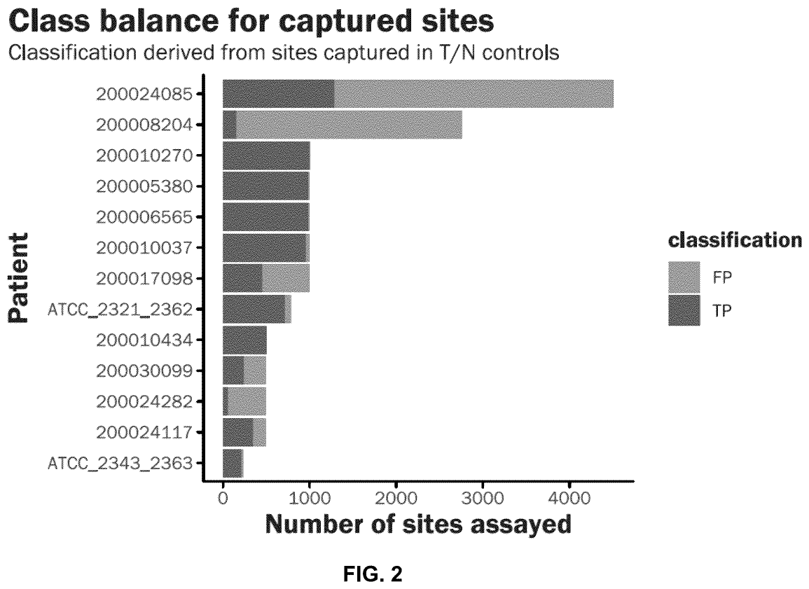

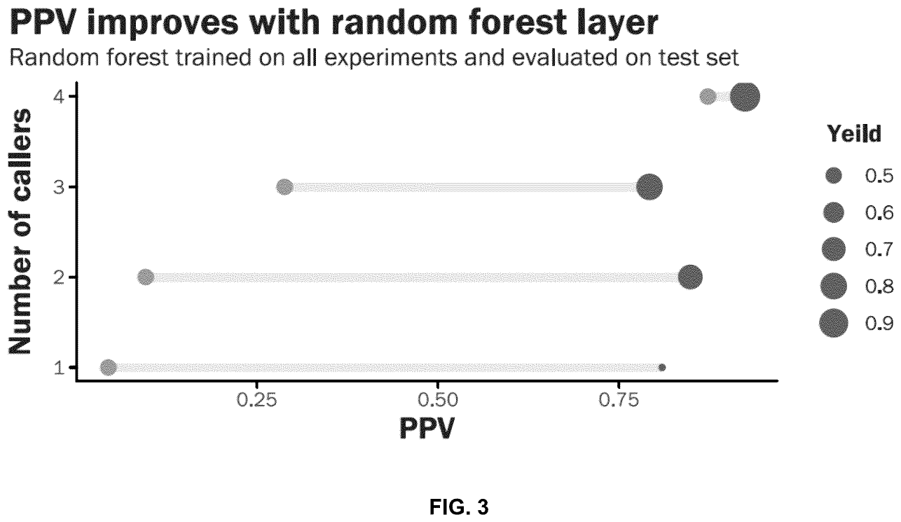

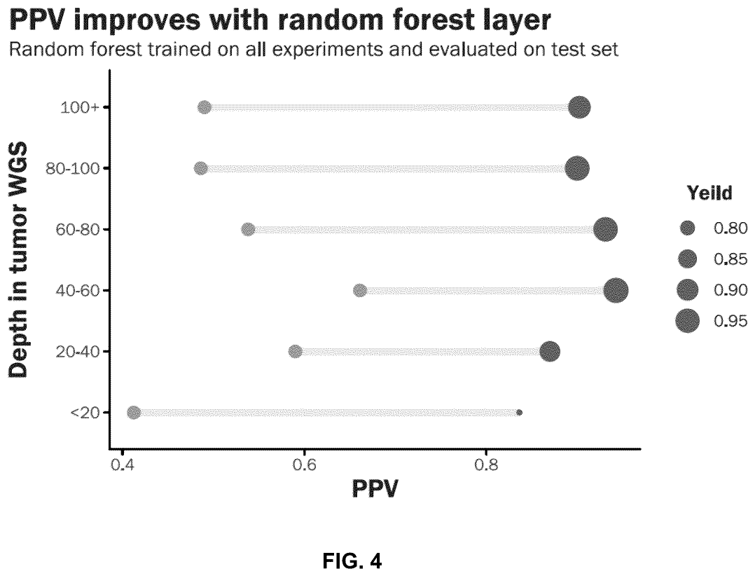

The classified dataset included approximately 15,000 target variants across 13 patients (FIG. 2). The classes, true positive (TP) variant calls (i.e., true somatic variant) and false positive (FP) variant calls (i.e., false somatic variant), were evenly distributed. The targets included in this dataset span a range of somatic variant call confidence, measured by the number of somatic variant callers in the ensemble that returned a positive somatic variant call. Two RF classifiers were trained: one classifier used targets from all confidence levels and a second classifier with only targets with the highest level of confidence (somatic variants called by all callers). Each classifier was trained on a subset of the data and then validated on the data that was held out of training. The positive predictive value (PPV) for targets that were called by the ensemble of somatic variant callers only versus those classified at TP by the RF classifier was assessed by dividing the number of TP targets by the total number of targets (Table 1). For both “all” and “high” confidence variant calls, inclusion of the RF classifier improved the PPV, effectively reducing the error in target selection when incorporating the RF classifier. The improvements observed in PPV were most substantial in the more difficult calling condition such as slow confidence (FIG. 3) and low whole genome sequencing depth (FIG. 4).

TABLE 1
Positive Predictive value (PPV) calculated with and
without applying the random forest (RF) classifier.

| Confidence variant calls | RF | n (total) | True Positive | PPV |
|---|---|---|---|---|
| All | False | 3371 | 2652 | 0.787 |
| All | True | 2707 | 2507 | 0.926 |
| High | False | 2962 | 2592 | 0.875 |
| High | True | 2646 | 2463 | 0.931 |

### Example 3: Optimization of Panel Selection to Increase the Member Molecules Sequenced Per Target

Whole genome sequencing was performed on 31 clinical sample sets of a FFPE tumor sample and FFPE normal sample per set. Somatic variants were called with an ensemble of somatic variant callers. A panel of targets was selected for each sample based on these somatic variant calls and the panel was constructed.

Each panel was used for hybrid capture and subsequent sequencing of its match tumor and normal sample was performed. Allele counts at each target were collected. Targets for which the alternate allele was detected in the tumor sample and not the normal sample were classified as true somatic variants. Targets for which the alternate allele was not detected in the tumor sample or was detected in the normal sample were classified as false somatic variants.

Each panel was additionally used for hybrid capture and subsequent sequencing of a patient-matched plasma-derived cfDNA sample. The total molecular depth obtained for each target was calculated and normalized by the median depth for the sample.

Features were calculated for each target in each sample, such as, GC content, sequence entropy and mappability (i.e., the number of blast hits returned for a region of sequence proximal to and inclusive of the target). Targets were binned by the calculated feature and both a positive predictive value (PPV), based on the variant classifications described above, and median relative target depth was calculated for that bin. One thousand simulations of target selection were run using two approaches. First, 1000 targets were randomly selected (random panel). Alternatively, 2000 targets were randomly selected, then each target was ranked by relative depth * PPV for the corresponding feature bin and the top 1000 ranked targets were selected (optimized panel). For each panel, the true effective panel depth was calculated by summing the total observed depth over each target that was confirmed a true positive somatic variant.

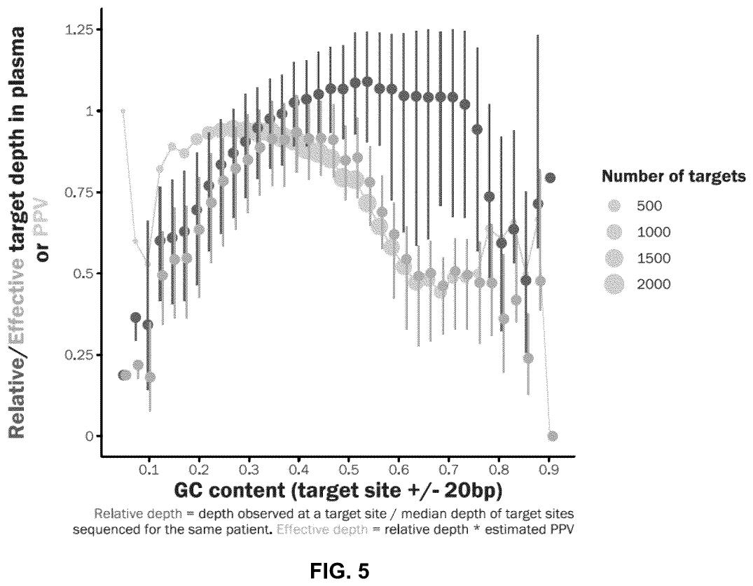

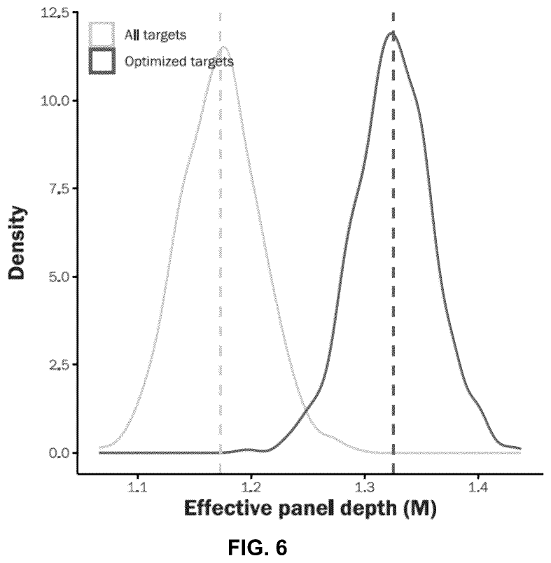

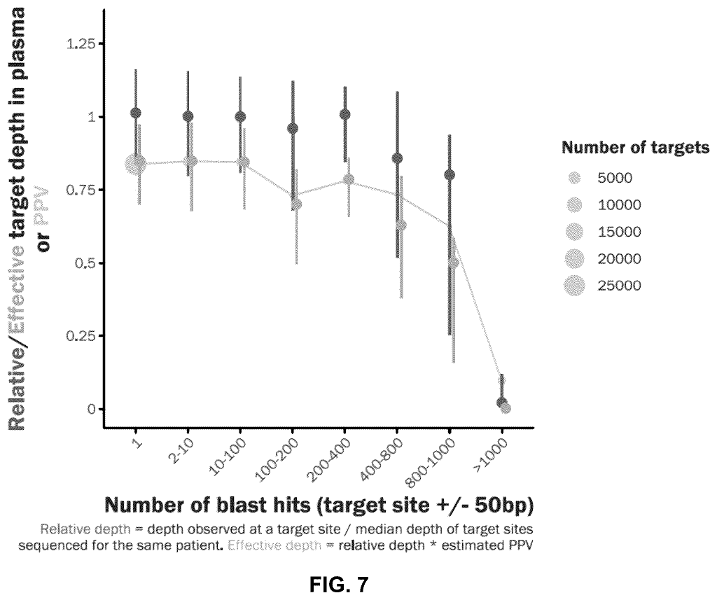

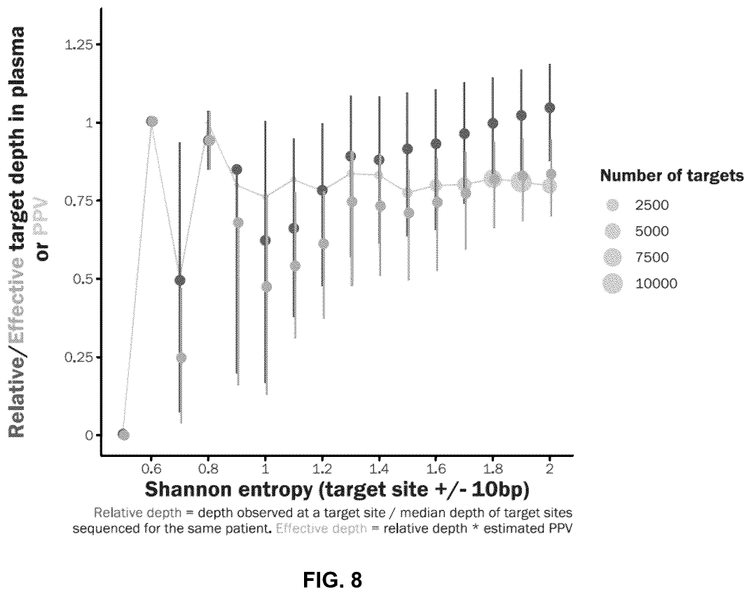

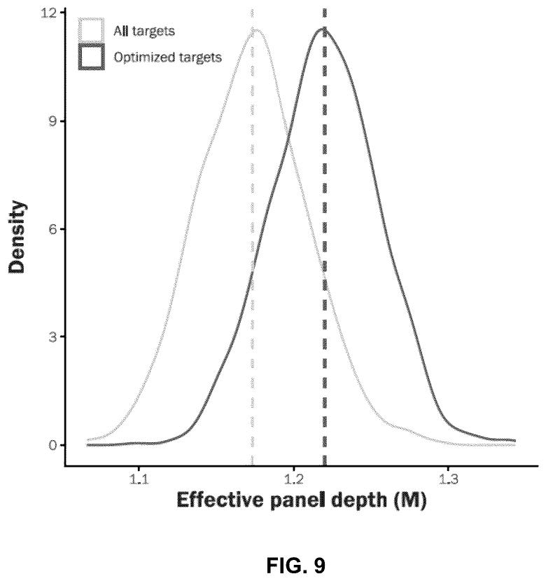

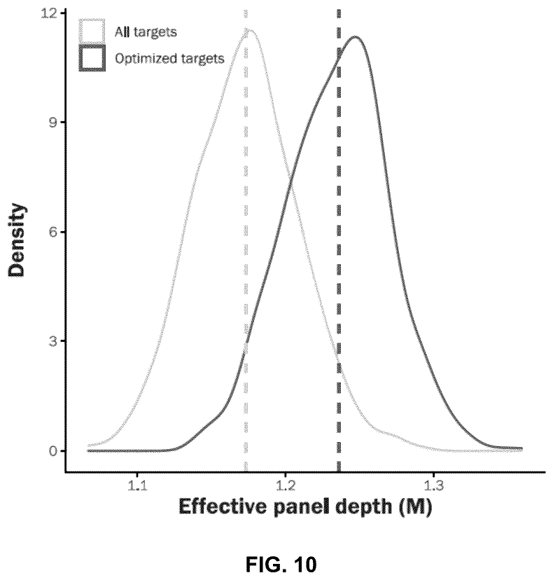

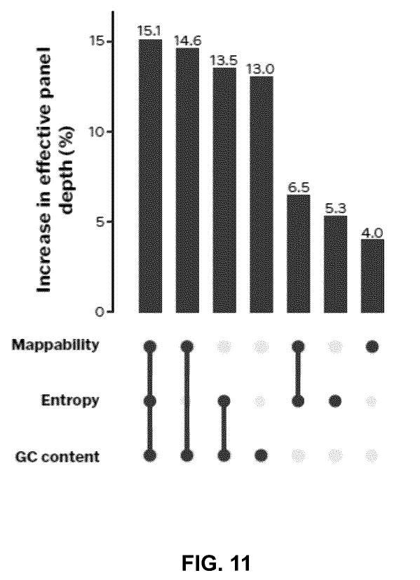

The GC content has a significant impact on sequence depth, but had an unexpected relationship with PPV, where false positive somatic variant calls are more common in regions with high GC content (FIG. 5). This relationship nullifies any benefit from optimizing for target depth based on GC without also considering PPV of the target as described in Example 2. When both GC and PPV were considered together, optimized panels resulted in approximately 13% greater effective panel depth (FIG. 6). Alternatively, less complex relationships were found using mappability (FIG. 7) and sequence entropy (FIG. 8) in concert with PPV. Optimization based on mappability and PPV resulted in a 4% increase in effective panel depth (FIG. 9) and a 5.3% increase based on sequence entropy and PPV (FIG. 10). In this experiment, use of all three sequence features in combination with PPV showed a 15.1% increase in the effective panel depth (FIG. 11).

### Example 4: Improved Sensitivity by Ranking Candidate Targets

Whole genome sequencing was performed on A pair of matched tumor and normal cell lines were analyzed by whole genome sequencing and candidate somatic variant targets were identified. These targets were ranked using the equation:

$$( p - error\_rate \_prior ) \times predicted\_depth \times PPV$$

where, p the expected alternate allele frequency was,

$$p = ( ( TF \times CNT ) / ( TF \times CNT + ( 1 - TF ) \times CNN ) ) \times ABT \times CF$$

where, error rate priors are obtained from historical control target data on cfDNA samples and was specific to the mutation type (e.g., C>T, T>A), predicted depth was a correction factor obtained from historical target depth data on cfDNA and are specific to the mutation class (e.g., SNV or indel), PPV was an estimate of the positive predictive value of the target, TF represents a tumor fraction that can be optimized depending on the desired TF range for the assay (set to 10e-5 for this experiment), CNT was the copy number of the site in the tumor, CNN was the copy number of the site in the normal (set to 2 for this experiment), ABT was the observed somatic variant allele balance in the tumor, CF was a correction factor for the ABT obtained from historical target allele balance data on captured tumor samples that accounts for target capture allele bias and was specific to the mutation class and length.

One thousand of the top-ranking targets were selected and oligonucleotide probes targeting those variants were manufactured. The tumor and normal cell line DNA was sheared to mimic cell-free DNA and mixed at various ratios to obtain tumor DNA concentrations ranging from 5-100 ppm. Five replicates of contrived samples at each concentration using two sets of reagent lots underwent sequencing library preparation, including UMIs, hybridization capture using the probe panel and targeted sequencing. Sequence data was deduplicated using the combined family read approach.

Prior to analysis, artificial panels were subset from the original 1000 targets. One panel contained only the top 250 highest ranking targets and a second panel contained the bottom 250 ranking targets. The target data for each sub-panel was analyzed using a likelihood model to estimate a most likely tumor fraction for the sample and a log likelihood ratio (LLR) of that tumor fraction relative to a null hypothesis that no tumor DNA was present. The LLR represents the level of confidence the tumor DNA was detected. As such, a detection threshold based on the LLR was imposed and sensitivity was calculated at each concentration level.

Sensitivity results are compiled for the top and bottom ranking targets and results presented in Tables 2 and 3, respectively. A Limit of Detection (LOD) was defined for each set of targets as the maximum of the within-reagent lot LODs, where LOD is the concentration at which ≥95% of samples are detected. The top ranking targets had an LOD of 20 ppm and the bottom ranking targets had an LOD of 50 ppm, demonstrating that ranking targets using the method described above improves the sensitivity of the assay.

TABLE 2
Sensitivity results for the top 250 ranking targets.

| | |
|---|---|
| Reagent lot 1 | Reagent lot 2 |

| Concentration | | Hit rate | | Hit rate |
|---|---|---|---|---|
| (ppm) | n | (sensitivity) | n | (sensitivity) |
| 5 | 10 | 0.2 | 10 | 0.6 |
| 10 | 9 | 0.556 | 10 | 0.7 |
| 20 | 9 | 1 | 9 | 1 |
| 50 | 8 | 1 | 9 | 1 |
| 100 | 10 | 1 | 7 | 1 |
| | | | LOD | 20 |

TABLE 3
Sensitivity results for the bottom 250 ranking targets.

| | |
|---|---|
| Reagent lot 1 | Reagent lot 2 |

| Concentration | | Hit rate | | Hit rate |
|---|---|---|---|---|
| (ppm) | n | (sensitivity) | n | (sensitivity) |
| 5 | 10 | 0.2 | 10 | 0.3 |
| 10 | 9 | 0.667 | 10 | 0.8 |
| 20 | 9 | 0.778 | 9 | 1 |
| 50 | 8 | 1 | 9 | 1 |
| 100 | 10 | 1 | 7 | 1 |
| | | | LOD | 50 |

It is understood that the examples and embodiments described herein are for illustrative purposes only and that various modifications or changes in light thereof will be suggested to persons skilled in the art and are to be included within the spirit and purview of this application and scope of the appended claims. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

1. A method of preparing a probe set for enriching circulating tumor DNA (ctDNA) from a sample, comprising:
sequencing DNA obtained from a tumor sample from a cancer patient and sequencing DNA obtained from a non-tumor sample from the cancer patient, thereby obtaining sequences of DNA from the tumor sample and sequences of DNA from the non-tumor sample;
determining somatic variants based on differences between sequences of DNA from the tumor sample and sequences of DNA from the non-tumor sample;
calculating, using a computer processor, an expected variant allele frequency based on copy number and allele balance of each of the somatic variants;
selecting a subset of somatic variants for which the expected variant allele frequency is above a reference threshold; and
preparing a probe set comprising a plurality of oligonucleotides, wherein each oligonucleotide in the plurality of oligonucleotides comprises a nucleic acid sequence that is capable of hybridizing to a DNA fragment comprising one of the subset of somatic variants for which the expected variant allele frequency is above a reference threshold.

2. The method of claim 2 further comprising:
sequencing cell-free DNA (cfDNA) extracted from at least one further sample of blood, plasma, or serums from the cancer patient, thereby obtaining a plurality of sequence reads; and
detecting in the plurality of sequence reads a sequence read comprising any one of the subset of somatic variants, thereby detecting the presence of ctDNA in the at least one further sample.

3. (canceled)

4. A method comprising:
(a) retrieving, by a processor, data associated with a tumor sample and a non-tumor sample from a cancer patient;
(b) generating, by the processor, a training data set comprising sequenced DNA from the tumor sample, sequenced DNA from the non-tumor sample, and somatic variants based on differences between sequences of DNA from the tumor sample and sequences of DNA from the non-tumor sample; and
(c) training, by the processor, a machine learning model using the training dataset, such that the machine learning model is configured to ingest data associated with a second tumor sample and a second non-tumor sample from a patient and predict a subset of somatic variants that are suitable for isolating ctDNA for the patient.

5. (canceled)

6. A method of detecting circulating tumor DNA (ctDNA) in a sample, comprising:
sequencing DNA obtained from a tumor sample from a cancer patient and sequencing DNA obtained from a non-tumor sample from the cancer patient, thereby obtaining sequences of DNA from the tumor sample and sequences of DNA from the non-tumor sample;
determining a set of somatic variants based on differences between sequences of DNA from the tumor sample and sequences of DNA from the non-tumor sample;
selecting a subset of somatic variants that are suitable for enriching ctDNA by:
(i) calculating, using a computer processor, an expected variant allele frequency based on copy number and allele balance of each of the somatic variants in the set of somatic variants, and selecting somatic variants for which the expected variant allele frequency is above a reference threshold,
(ii) determining, based on a plurality of features of the set of somatic variants, a subset of somatic variants that are suitable for enriching ctDNA, wherein the plurality of features are selected by a machine learning model trained with genomic data, or
(iii) a combination of (i) and (ii); and
sequencing cell-free DNA (cfDNA) extracted from at least one further sample of blood, plasma, or serums from the cancer patient, thereby obtaining a plurality of sequence reads; and
detecting in the plurality of sequence read a sequence read comprising any one of the subset of somatic variants, thereby detecting the presence of ctDNA in the at least one further sample.

7.-8. (canceled)

9. The method of claim 2, further comprising enriching the extracted cfDNA prior to sequencing by contacting the extracted cfDNA with a plurality of oligonucleotides, wherein each oligonucleotide in the plurality of oligonucleotides comprises a nucleic acid sequence that is capable of hybridizing to a DNA fragment comprising one of the somatic variants, thereby obtaining a ctDNA-enriched fraction;
optionally wherein the enriching comprises (i) hybrid capture-based enrichment, (ii) PCR-target enrichment, or (iii) on-sequencer enrichment.

10. The method of claim 2, wherein the sequencing comprises whole genome sequencing or targeted sequencing.

11. The method of claim 10, wherein the targeted sequencing comprises sequencing of introns, exons, intergenic regions, or a combination thereof.

12. The method of claim 2, wherein the subset of somatic variants comprises at least 10, at least 50, at least 100, at least 150, at least 200, at least 250, or at least 500 tumor-specific somatic mutations.

13. The method of claim 12, wherein the subset of somatic variants comprises one or more somatic mutations selected from SNVs, insertions, deletions, and translocations.

14. The method of claim 2 further comprising determining a tumor fraction.

15. (canceled)

16. The method of claim 2, wherein the tumor sample comprises a solid tumor biopsy or a fluid sample.

17. The method of claim 16, wherein the fluid sample is selected from blood, blood plasma, blood serum, urine, saliva, and cerebral spinal fluid (CSF).

18. The method of claim 2, wherein the non-tumor sample comprises a tissue sample matched to a tissue of origin of the tumor sample.

19. (canceled)

20. The method of claim 2, wherein the patient has completed at least one cancer treatment prior to obtaining the tumor sample and the non-tumor sample.

21. (canceled)

22. The method of claim 2 further comprising repeating (f)-(i) with a second, third, fourth, fifth, sixth, seventh, eight, ninth, or tenth further sample of blood, plasma, or serum at successive time points.

23. The method of claim 22, wherein (f)-(i) are repeated one or more times while the patient is in remission or is undergoing treatment for the cancer.

24. (canceled)

25. The method of claim 22, wherein (f)-(i) are repeated one or more times coinciding with or prior to surgery; following, during, or prior to administration of chemotherapy; following, during, or prior to radiation therapy; following, during, or prior to administration of an immunotherapy; following, during, or prior to administration of a cell therapy; or following, during, or prior to administration of a biologic therapy.

26.-29. (canceled)

30. The method of claim 2, wherein the tumor is selected from adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor.

31. The method of claim 2, wherein selecting a subset of somatic variants comprises subtracting an expected error rate from the expected variant allele frequency.

32. The method of claim 33, wherein the genomic data comprises DNA sequencing data, whole genome sequencing data, whole exome sequencing data, targeted genomic sequencing data, cfDNA sequencing data, chromatin immunoprecipitation sequencing data, reference genome data, transcriptomics data, epigenomics data, or proteomics data.

33. A method of selecting tumor-specific somatic mutations for preparing a probe set for enriching circulating tumor DNA (ctDNA) from a sample, comprising:
sequencing DNA obtained from a tumor sample from a cancer patient and sequencing DNA obtained from a non-tumor sample from the cancer patient, thereby obtaining sequences of DNA from the tumor sample and sequences of DNA from the non-tumor sample;
determining a set of somatic variants based on differences between sequences of DNA from the tumor sample and sequences of DNA from the non-tumor sample; and
selecting a subset of tumor-specific somatic variants that are suitable for enriching ctDNA by:
(i) calculating, using a computer processor, an expected variant allele frequency based on copy number and allele balance of each of the somatic variants in the set of somatic variants, and selecting tumor-specific somatic variants for which the expected variant allele frequency is above a reference threshold,
(ii) determining, based on a plurality of features of the set of somatic variants, a subset of tumor-specific somatic variants, wherein the plurality of features are selected by a machine learning model trained with genomic data, or
(iii) a combination of (i) and (ii).
